(19) United States
(12) Patent Application Publication
(10) Pub. No.: US 2011/0152204 A1
(43) Pub. Date: Jun. 23, 2011

(54) Treatment of Obesity or Diabetes with Bile Acid Sequestrants

(75) Inventors: Bronislava GEDULIN, Del Mar, CA (US); Howard E. Greene, JR., Frankfort, MI (US)

(73) Assignee: Satiogen Pharmaceuticals, Inc., San Diego, CA (US)

(21) Appl. No.: 12/969,388

(22) Filed: Dec. 15, 2010

Related U.S. Application Data
(60) Provisional application No. 61/288,134, filed on Dec. 18, 2009.

(51) Int. Cl. A61K 31/56; A61K 31/09; A61K 31/14; A61K 31/131; A61K 31/132; A61K 31/20; A61K 31/155; A61P 3/10; A61P 3/04

(52) U.S. Cl. 514/22; 514/642; 514/671; 514/169; 514/558; 514/635

(57) ABSTRACT

Provided herein are methods of treating obesity and diabetes with labile bile acid sequestrants. An effective amount of a labile bile acid sequestrant may be orally administered to an obese or diabetic individual. A labile bile acid sequestrant provided herein may have a low affinity in the colon or rectum of a human for at least one bile acid or bile acid mimic that stimulates L-cells. A labile bile acid sequestrant may be a non-systemic labile bile acid sequestrant.

### CROSS-REFERENCE TO RELATED APPLICATIONS

This application claims the benefit under 35 U.S.C. §119(e) of U.S. Provisional Application No. 61/288,134, filed Dec. 18, 2009, which is incorporated herein by reference in its entirety.

### BACKGROUND OF THE INVENTION

Obesity is a medical condition affecting numerous humans in developed countries throughout the world, and is associated with or induces other diseases or conditions. In particular, obesity is a serious risk factor for diseases and conditions such as diabetes, hypertension, gallbladder disease, cancer, polycystic ovary disease and arteriosclerosis and can contribute to elevated levels of cholesterol in the blood. In addition, increased body weight due to obesity places a burden on joints causing arthritis, pain, and stiffness. Overeating and obesity have become a problem in the general population. Consequently, there is interest in losing weight, reducing weight, and/or maintaining a healthy body weight and lifestyle.

Type 2 diabetes is a chronic disease that is marked by high levels of sugar in the blood. About 23.6 million people (7.8%) of people in the United States suffer from diabetes. An additional 57 million people are pre-diabetic. Diabetes was the seventh leading cause of death in the United States in 2006. As such, there is an unmet need for treatment of obesity and diabetes.

### SUMMARY OF THE INVENTION

Provided in certain embodiments herein is a method of treating obesity and/or diabetes in an individual comprising orally administering to an individual in need thereof an effective amount of a labile bile acid sequestrant, wherein the labile bile acid sequestrant has a low affinity in the colon or rectum of the individual for at least one bile acid that stimulates L-cells. In some embodiments, provided herein is a labile bile acid sequestrant for use in the treatment of obesity and/or diabetes in an individual, wherein the labile bile acid sequestrant has a low affinity in the colon or rectum of the individual for at least one bile acid that stimulates L-cells. In some embodiments, the individual is a human. In some embodiments, a labile bile acid sequestrant provided herein releases a bile acid in the colon or the rectum of a human. In some embodiments, a labile bile acid sequestrant provided herein does not sequester a bile acid for excretion or elimination in feces. In some embodiments, a labile bile acid sequestrant provided herein is a non-systemic labile bile acid sequestrant. In some embodiments, non-systemic labile bile acid sequestrant is less than 1%, 2%, 3%, 4%, 5%, 6%, 7%, 8%, 9%, 10%, 15%, 20%, 25%, 30%, 35%, 40%, 45% absorbed systemically.

In certain embodiments, provided herein is a method of treating obesity and/or diabetes in an individual comprising orally administering to an individual in need thereof an effective amount of a labile bile acid sequestrant that is a carrier or a delivery agent for an exogenous bile acid, a bile salt, a bile acid mimic, a free fatty acids or free fatty acids mimic. In some embodiments, a labile bile acid sequestrant delivers an exogenous bile acid, a bile salt, a bile acid mimic, a free fatty acids or free fatty acids mimic to the colon or the rectum of a human. In some embodiments, a labile bile acid sequestrant releases an exogenous bile acid, a bile salt, a bile acid mimic, a free fatty acids or free fatty acids mimic in the colon or the rectum of a human. In some embodiments, a labile bile acid sequestrant is a non-systemic labile bile acid sequestrant. In some embodiments, non-systemic labile bile acid sequestrants provided herein deliver less than 45%, less than 40%, less than 35%, less than 30%, less than 25%, less than 20%, less than 15%, less than 10%, less than 5%, less than 4%, less than 3%, less than 2%, or less than 1% w/w of the bile acid sequestrant systemically.

In certain embodiments, a labile bile acid sequestrant is a non-systemic bile acid sequestrant that is synthesized or manufactured with an exogenous bile acid, a bile salt, a bile acid mimic, or a bile salt mimic, free fatty acids, or a free fatty acids mimic attached, linked, or conjugated to the labile bile acid sequestrant. In some embodiments, a labile bile acid sequestrant is covalently linked to an exogenous bile acid, a bile salt, a bile acid mimic, or a bile salt mimic, free fatty acids, or a free fatty acids mimic. In some embodiments, a labile bile acid sequestrant is attached to an exogenous bile acid, a bile salt, a bile acid mimic, or a bile salt mimic, free fatty acids, or a free fatty acids mimic by a linker. In some embodiments, the linker is designed to be degraded or severed in the colon or the rectum. In some embodiments, an exogenous bile acid, a bile salt, a bile acid mimic, or a bile salt mimic, free fatty acids, or a free fatty acids mimic is released from a labile bile acid sequestrant in the colon or the rectum via a time-dependent, pH-dependent, or intestinal motility-dependent mechanism.

In some embodiments, a labile bile acid sequestrant is an enzyme dependent bile acid sequestrant. In certain embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

In certain embodiments, a labile bile acid sequestrant is a time dependent bile acid sequestrant. In some embodiments, a labile bile acid sequestrant releases a bile acid or is degraded after 1, 2, 3, 4, 5, 6, 7, 8, 9, or 10 seconds of sequestration. In some embodiments, a labile bile acid sequestrant releases a bile acid or is degraded after 15, 20, 25, 30, 35, 40, 45, 50, or 55 seconds of sequestration. In some embodiments, a labile bile acid sequestrant releases a bile acid or is degraded after 1, 2, 3, 4, 5, 6, 7, 8, 9, or 10 minutes of sequestration. In some embodiments, a labile bile acid sequestrant releases a bile acid or is degraded after about 15, 20, 25, 30, 35, 45, 50, or 55 minutes of sequestration. In some embodiments, a labile bile acid sequestrant releases a bile acid or is degraded after about 1, 2, 3, 4, 5, 6, 7, 8, 9, 10, 11, 12, 13, 14, 15, 16, 17, 18, 19, 20, 21, 22, 23, or 24 hours of sequestration. In some embodiments, a labile bile acid sequestrant releases a bile acid or is degraded after 1, 2, or 3 days of sequestration.

In some embodiments, the labile bile acid sequestrant has a low affinity for bile acid. In certain embodiments, the labile bile acid sequestrant has a high affinity for a primary bile acid and a low affinity for a secondary bile acid.

In some embodiments, the labile bile acid sequestrant is a pH dependent bile acid sequestrant. In certain embodiments, the pH dependent bile acid sequestrant has a high affinity for bile acid at a pH of 6 or below and a low affinity for bile acid at a pH above 6. In certain embodiments, the pH dependent bile acid sequestrant has a high affinity for bile acid at a pH of 6.5 or below and a low affinity for bile acid at a pH above 6.5. In certain embodiments, the pH dependent bile acid sequestrant has a high affinity for bile acid at a pH of 7 or below and a low affinity for bile acid at a pH above 7. In certain embodiments, the pH dependent bile acid sequestrant has a high affinity for bile acid at a pH of 7.1 or below and a low affinity for bile acid at a pH above 7.1. In certain embodiments, the pH dependent bile acid sequestrant has a high affinity for bile acid at a pH of 7.2 or below and a low affinity for bile acid at a pH above 7.2. In certain embodiments, the pH dependent bile acid sequestrant has a high affinity for bile acid at a pH of 7.3 or below and a low affinity for bile acid at a pH above 7.3. In certain embodiments, the pH dependent bile acid sequestrant has a high affinity for bile acid at a pH of 7.4 or below and a low affinity for bile acid at a pH above 7.4. In certain embodiments, the pH dependent bile acid sequestrant has a high affinity for bile acid at a pH of 7.5 or below and a low affinity for bile acid at a pH above 7.5. In certain embodiments, the pH dependent bile acid sequestrant has a high affinity for bile acid at a pH of 7.6 or below and a low affinity for bile acid at a pH above 7.6. In certain embodiments, the pH dependent bile acid sequestrant has a high affinity for bile acid at a pH of 7.7 or below and a low affinity for bile acid at a pH above 7.7. In certain embodiments, the pH dependent bile acid sequestrant has a high affinity for bile acid at a pH of 7.8 or below and a low affinity for bile acid at a pH above 7.8. In some embodiments, the pH dependent bile acid sequestrant degrades at a pH above 6. In some embodiments, the pH dependent bile acid sequestrant degrades at a pH above 6.5. In some embodiments, the pH dependent bile acid sequestrant degrades at a pH above 7. In some embodiments, the pH dependent bile acid sequestrant degrades at a pH above 7.1. In some embodiments, the pH dependent bile acid sequestrant degrades at a pH above 7.2. In some embodiments, the pH dependent bile acid sequestrant degrades at a pH above 7.3. In some embodiments, the pH dependent bile acid sequestrant degrades at a pH above 7.4. In some embodiments, the pH dependent bile acid sequestrant degrades at a pH above 7.5. In some embodiments, the pH dependent bile acid sequestrant degrades at a pH above 7.6. In some embodiments, the pH dependent bile acid sequestrant degrades at a pH above 7.7. In some embodiments, the pH dependent bile acid sequestrant degrades at a pH above 7.8. In some embodiments, the pH dependent bile acid sequestrant degrades at a pH above 7.9.

In certain embodiments, the labile bile acid sequestrant is lignin or a modified lignin. In some embodiments, the labile bile acid sequestrant is a polycationic polymer or copolymer. In certain embodiments, the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof.

In some embodiments, an individual treated according to a method described herein is an obese or morbidly overweight individual. In certain embodiments, the individual is a diabetic individual. In some embodiments, the individual is a non-diabetic individual.

In certain embodiments, levels of GLP-1 in the blood and/or plasma of an individual treated according to a method described herein are increased by about 2 times to about 6 times or by about 2 times to about 10 times the level of GLP-1 in the blood and/or plasma of the individual prior to administration of the bile acid sequestrant. In certain embodiments, levels of GLP-1 in the blood and/or plasma of an individual treated according to a method described herein are increased by about 10%, 20%, 30%, 40%, 50%, 60%, 70%, 80%, 90%, 100%, 150%, 200%, 300%, 400%, 500%, 600%, 700%, 800%, 900%, or 1000% of the level of GLP-1 in the blood and/or plasma of the individual prior to administration of the bile acid sequestrant. In some embodiments, levels of post-prandial glucose in the blood and/or plasma of an individual treated according to a method described herein are reduced by at least 5%, 10%, 15%, 20%, 25%, 30%, 35%, 40%, 45%, 50%, 55%, 60%, 65%, 70%, 75%, 80%, 85%, 90%, 95%, or 100% compared to the level of glucose in the blood and/or plasma of the individual prior to administration of the bile acid sequestrant. In some embodiments, levels of post-prandial glucose in the blood and/or plasma of an individual treated according to a method described herein are reduced by at least 30% compared to the level of glucose in the blood and/or plasma of the individual prior to administration of the bile acid sequestrant. In certain embodiments, reduced blood and/or plasma glucose levels in an individual treated according to a method described herein are maintained for at least 1, 2, 3, 4, 5, 6, 7, 8, 9, 10, 11, 12, 13, 14, 15, 16, 17, 18, 19, 20, 21, 22, 23, or 24 hours compared to blood and/or plasma glucose levels in the individual prior to administration of the bile acid sequestrant. In certain embodiments, reduced blood and/or plasma glucose levels in an individual treated according to a method described herein are maintained for at least 24 hours compared to blood and/or plasma glucose levels in the individual prior to administration of the bile acid sequestrant. In certain embodiments, reduced blood and/or plasma glucose levels in an individual treated according to a method described herein are maintained for at least 1, 2, 3, 4, or 5 days compared to blood and/or plasma glucose levels in the individual prior to administration of the bile acid sequestrant. In some embodiments, a method described herein enhances enteroendocrine peptide secretion in an individual in need thereof.

In some embodiments, the labile bile acid sequestrant transports bile acids from the ileum, the duodenum, the jejenum, and/or appear ileum of the individual to the colon and/or rectum of the individual. In certain embodiments, a labile bile acid sequestrant sequesters bile acid and/or salts thereof in the small intestine of the individual and releases the bile acids and/or salts thereof into the colon and/or rectum of the individual. In some embodiments, a bile acid sequestrant is administered as an enterically coated formulation.

In certain embodiments, an effective amount of bile acid sequestrant administered in a method described herein is an amount sufficient to provide a bile acid concentration in the colon of greater than 3 mM. In some embodiments, the effective amount of bile acid sequestrant is an amount sufficient to provide a bile acid concentration in the colon of greater than 5 mM. In some embodiments, the effective amount of bile acid sequestrant is an amount sufficient to provide a bile acid concentration in the colon or the rectum of greater than 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 mM.

In some embodiments, any method described herein further comprising administration of a second agent selected from an ASBT inhibitor, metformin, an incretin mimetic, symlin, leptin, a DPP-IV inhibitor, a TGR5 agonist, a GPR119 agonist, a GPR120 agonist, a GPR40 agonist, a GPR43 agonist, and a GPR154 agonist. In specific embodiments, the bile acid sequestrant is administered in combination with an effective amount of DPP-IV inhibitor. In other specific embodiments, the bile acid sequestrant is administered in combination with an effective amount of symlin, an appetite suppressing symlin analog, an amylin modulator (e.g., up-regulator), leptin, an appetite suppressing leptin analog, or a leptin modulator (e.g., up-regulator).

In certain embodiments, the bile acid sequestrant is administered before ingestion of food. In some embodiments, the bile acid sequestrant is administered less than about 60 minutes before ingestion of food. In certain embodiments, the bile acid sequestrant is administered less than about 30 minutes before ingestion of food. In certain embodiments, the bile acid sequestrant is administered less than about 25, 20, 10, 5, or 1 minute before ingestion of food. In certain embodiments, the bile acid sequestrant is administered less than about 24, 20, 16, 12, 10, 9, 8, 7, 6, 5, 4, 3, 2, or 1 hour before ingestion of food. In some embodiments, the administered dose of the bile acid sequestrant is from about 0.001 mg/kg to about 100 mg/kg. In certain embodiments, the administered dose of the bile acid sequestrant is from about 0.001 mg/kg to about 50 mg/kg. In certain embodiments, the administered dose of the bile acid sequestrant is about 0.001, 0.005, 0.010, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 mg/kg.

In some embodiments, provided herein is the use of a bile acid sequestrant in the manufacture of a medicament in any method of treatment as described herein.

Provided in certain embodiments herein is a pharmaceutical composition comprising a therapeutically effective amount of a labile bile acid sequestrant.

In some embodiments, the labile bile acid sequestrant is a pH dependent bile acid sequestrant. In certain embodiments, the pH dependent bile acid sequestrant has a high affinity for bile acid at a pH of 7 or below and a low affinity for bile acid at a pH above 7. In some embodiments, the pH dependent bile acid sequestrant degrades at a pH above 7. In some embodiments, the labile bile acid sequestrant is a pH dependent bile acid sequestrant. In certain embodiments, the pH dependent bile acid sequestrant has a high affinity for bile acid at a pH of 6 or below and a low affinity for bile acid at a pH above 6. In some embodiments, the pH dependent bile acid sequestrant degrades at a pH above 6.

In certain embodiments, the labile bile acid sequestrant is an enzyme dependent bile acid sequestrant. In some embodiments, the enzyme is a bacterial enzyme. In certain embodiments, the enzyme is a bacterial enzyme found in high concentration in human colon or rectum relative to the concentration found in the ileum.

In some embodiments, the labile bile acid sequestrant is a time dependent bile acid sequestrant. In certain embodiments, the labile bile acid sequestrant has a low affinity for bile acid.

In certain embodiments, the labile bile acid sequestrant is lignin or a modified lignin. In some embodiments, the labile bile acid sequestrant is a polycationic polymer or copolymer. In certain embodiments, the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates various bile acids (including salts thereof) that may be sequestered with a bile acid sequestrant herein. In some instances, a labile bile acid sequestrant may sequester one of these bile acids in the ileum, but release it in the colon and rectum.

FIG. 2 illustrates the normal GI distribution of PYY (pmol/g).

FIG. 3 illustrates the gastrointestinal pH profiles in normal human subjects.

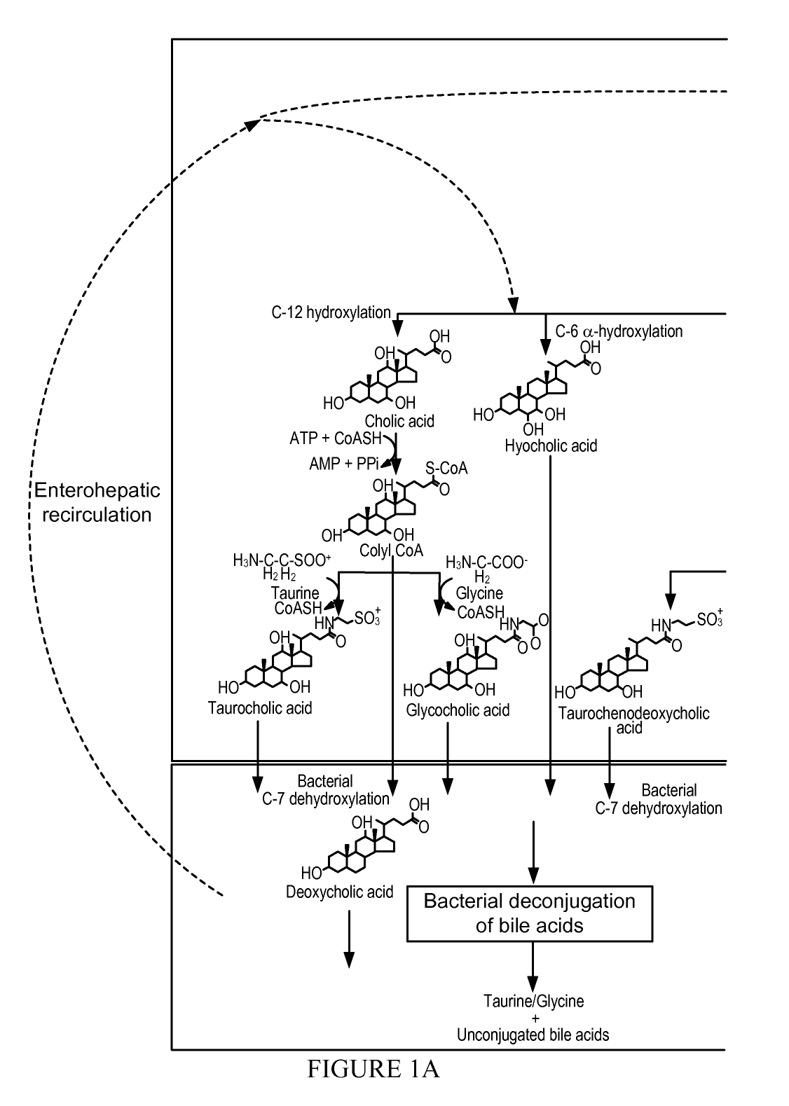

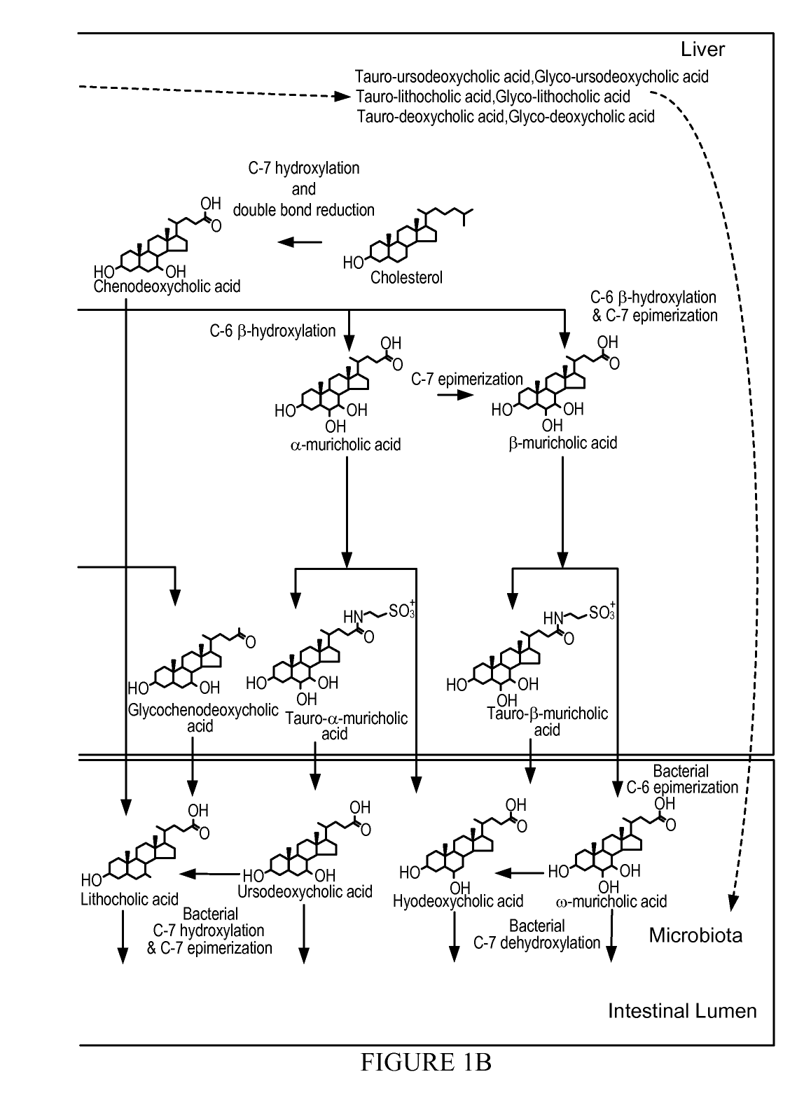

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Provided in some embodiments herein is a method of treating obesity or diabetes in an individual comprising orally administering to an individual in need thereof an effective amount of a bile acid sequestrant (e.g., labile bile acid sequestrant), wherein the bile acid sequestrant (e.g., labile bile acid sequestrant) has a low affinity in the colon or rectum of a human for at least one bile acid that stimulates L-cells. Described in certain embodiments herein is the use of a bile acid sequestrant (e.g., labile bile acid sequestrant) that are active in the gastrointestinal (GI) tract for induction of weight loss in an individual in need thereof.

In some instances, the delivery of a bile acid sequestrant (e.g., labile bile acid sequestrant) to the gastrointestinal tract (e.g., the colon and/or rectum via the small intestines) results in higher concentrations of bile salts in the lumen of the gastrointestinal tract or portions thereof (e.g., the distal small bowel and/or colon and/or rectum). In certain embodiments, the higher concentration of bile salts in the distal small bowel and/or colon and/or rectum modulates (e.g., enhances) the secretion of enteroendocrine peptides in the gastrointestinal tract. In some embodiments, the bile acid sequestrants (e.g., labile bile acid sequestrants) described herein enhance the secretion of enteroendocrine peptides (e.g., GLP-1, GLP-2, oxyntomodulin, PYY) from L-cells that are present in the gastrointestinal tract. In certain embodiments, the enhanced secretion of L-cell enteroendocrine peptides modulates (e.g., slows or inhibits) gastric emptying and gastric acid secretion. In certain instances the enhanced secretion of L-cell enteroendocrine peptides induces a feeling of satiety. In some embodiments, the enhanced secretion of L-cell enteroendocrine peptides reduces food intake.

Also described herein is the use of such bile acid sequestrants (e.g., labile bile acid sequestrants) for reducing or maintaining weight in individuals (e.g., individuals interested in losing weight, reducing weight, and/or maintaining a healthy body weight and lifestyle). In certain instances, the bile acid sequestrants (e.g., labile bile acid sequestrants) described herein are useful in the treatment of diseases or conditions that are modulated by enteroendocrine peptides secreted by L-cells of the gastrointestinal tract. In some instances, enhanced enteroendocrine peptide secretion (e.g., increase in GLP-1 secretion) reduces blood or plasma glucose levels. In some embodiments, the bile acid sequestrants (e.g., labile bile acid sequestrants) described herein are also useful in the treatment of metabolic disorders (e.g., diabetes, metabolic syndrome or the like) because they do not cause side effects (e.g., weight gain) that are associated with conventional therapies for metabolic disorders.

### Bile Acid Sequestrant

In certain embodiments, a bile acid sequestrant described herein is an agent that sequesters (e.g., absorbs or is charged with) bile acid, and/or the salts thereof. In various embodiments described herein, administration of the bile acid sequestrant to an individual according to any method described herein provides for sequestering of bile acid and/or salts thereof by the bile acid sequestrant in the small intestine, antrum, duodenum, jejunum, ileum, or any combination thereof.

In specific embodiments, the bile acid sequestrant is a labile bile acid sequestrant. In more specific embodiments, the labile bile acid sequestrant is an agent that sequesters (e.g., absorbs or is charged with) bile acid, and/or the salts thereof, in the small intestine, antrum, duodenum, jejunum, ileum, or any combination thereof, and subsequently releases at least a portion of the absorbed or charged bile acid, and/or salts thereof, at a point further in the gastrointestinal tract (e.g., the colon, ascending colon, sigmoid colon, distal colon, rectum, or any combination thereof). In specific embodiments, the labile bile acid sequestrant transports bile acids from the ileum of the individual to the colon and/or rectum of the individual. In some embodiments, a labile bile acid sequestrant described herein sequesters bile acid and/or salts thereof in the small intestine of the individual and releases the bile acids and/or salts thereof into the colon and/or rectum of the individual.

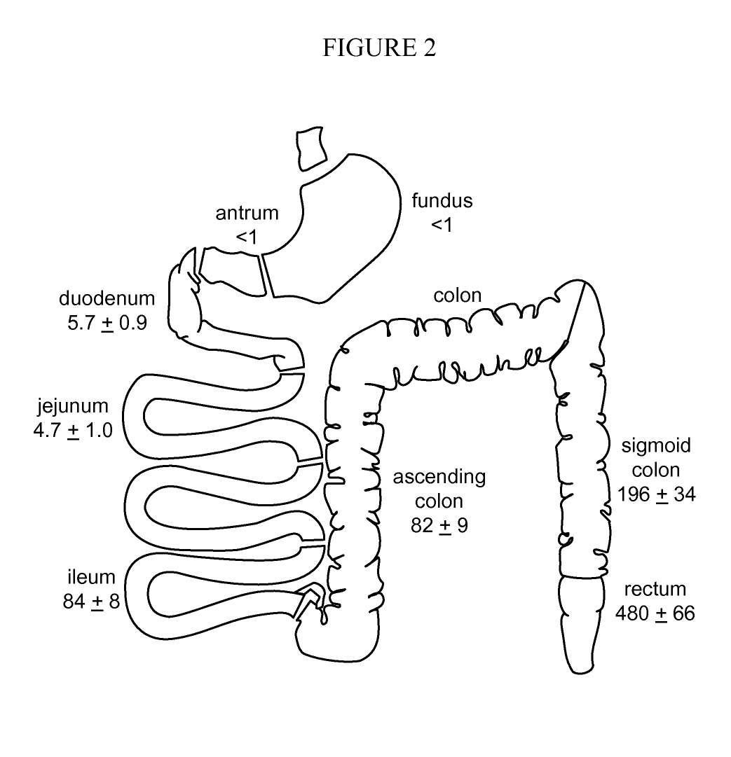

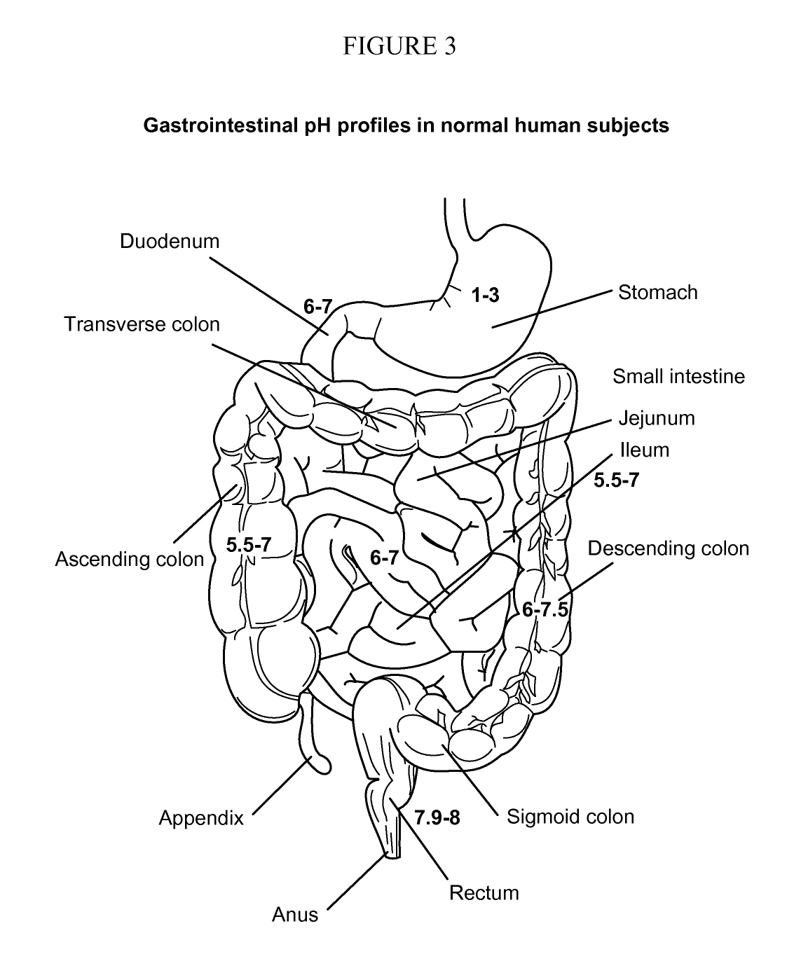

In certain instances, non-labile bile acid sequesterants are designed to reduce cholesterol in an individual. This is generally achieved by designing a bile acid sequestrant that binds bile acids as tightly as possible in order to remove the bile acids from the body and thereby drawdown the cholesterol pool. Labile bile acid sequestrants, on the other hand, do not bind the bile acids as tightly as possible because the labile bile acids are designed to deliver bile acids to the distal gut (e.g., colon and/or rectum). In certain instances, delivery of the bile acids to the distal gut (e.g., colon and/or rectum) stimulates L-cells and/or TGR5 receptors thereby providing therapeutic effect for obesity and/or diabetes. In some instances, this therapy is especially effective because L-cell concentrations rise significantly in the distal gut, with the results that bile acids have a much more potent secretory effect for metabolic hormones. FIG. 2 illustrates the presence of the metabolic hormone PYY in the gastrointestinal tract with significant increases in the distal gut.

In certain embodiments, a labile bile acid sequestrant described herein is an enzyme dependent bile acid sequestrant. In specific embodiments, the enzyme is a bacterial enzyme. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like.

In some embodiments, a labile bile acid sequestrant described herein is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time.

In certain embodiments, a labile bile acid sequestrant described herein is a bile acid sequestrant that has a low affinity for bile acid and/or salts thereof, thereby allowing the bile acid sequestrant to sequester bile acid and/or salts thereof in an environ where the bile acids and/or salts thereof are present in high concentration and release them in an environ wherein bile acids and/or salts thereof are present in a lower relative concentration. In some embodiments, a labile bile acid sequestrant described herein has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof.

In some embodiments, a labile bile acid sequestrant described herein is a pH dependent bile acid sequestrant. In some embodiments, the pH dependent bile acid sequestrant has a high affinity for bile acid at a pH of 6 or below and a low affinity for bile acid at a pH above 6. In certain embodiments, the pH dependent bile acid sequestrant degrades at a pH above 6.

In some embodiments, bile acid sequestrants (e.g., labile bile acid sequestrants) described herein include any compound, e.g., a macrostructured compound, that can sequester bile acids and/or salts thereof through any suitable mechanism. For example, in certain embodiments, bile acid sequestrants sequester bile acids and/or salts thereof through ionic interactions, polar interactions, static interactions, hydrophobic interactions, lipophilic interactions, hydrophilic interactions, steric interactions, or the like. In certain embodiments, macrostructured compounds sequester bile acids and/or sequestrants by trapping the bile acids and/or salts thereof in pockets of the macrostructured compounds and, optionally, other interactions, such as those described above. In some embodiments, bile acid sequestrants (e.g., labile bile acid sequestrants) include, by way of non-limiting example, lignin, modified lignin, polymers, polycationic polymers and copolymers, polymers and/or copolymers comprising anyone one or more of N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof, or any combination thereof.

### General Definitions

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, preventing, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that are optionally employed with the agents and methods described herein are found in sources e.g., Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “bile acid,” as used herein, includes steroid acids, and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate and the like. Taurocholic acid and/or taurocholate are referred to herein as TCA. Any reference to a bile acid used herein includes reference to a bile acid, one and only one bile acid, one or more bile acids, or to at least one bile acid. Therefore, the terms “bile acid,” “bile salt,” “bile acid/salt,” “bile acids,” “bile salts,” and “bile acids/salts” are, unless otherwise indicated, utilized interchangeably herein. Any reference to a bile acid used herein includes reference to a bile acid or a salt thereof. Furthermore, it is to be understood that as used herein, “bile acids” include bile acids conjugated to an amino acid (e.g., glycine or taurine). For example, the term “bile acid” includes cholic acid conjugated with either glycine or taurine: glycocholate and taurocholate, respectively (and salts thereof). Any reference to a bile acid used herein includes reference to an identical compound naturally or synthetically prepared. Furthermore, it is to be understood that any singular reference to a component (bile acid or otherwise) used herein includes reference to one and only one, one or more, or at least one of such components. Similarly, any plural reference to a component used herein includes reference to one and only one, one or more, or at least one of such components, unless otherwise noted.

The term “labile bile acid sequestrant” is a bile acid sequestrant that possesses a labile affinity for binding bile acids (i.e., bile acids and/or salts).

The term “colon,” as used herein, includes the cecum, ascending colon, hepatic flexure, splenic flexure, descending colon, and sigmoid.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

The term “ASBT inhibitor” refers to a compound that inhibits apical sodium-dependent bile transport or any recuperative bile salt transport. The term Apical Sodium-dependent Bile Transporter (ASBT) is used interchangeably with the term Ileal Bile Acid Transporter (IBAT).

The term “reducing food intake” refers to consumption of a lower amount of food by an individual undergoing any therapy described herein compared to the amount of food consumed in the absence of therapy.

The term “induction of satiety” or “inducing satiety” or “satiety” refers to a feeling of fullness and/or a reduction of the sensation of hunger.

The term “metabolic disorder” refers to any disorder that involves an alteration in the normal metabolism of carbohydrates, lipids, proteins, nucleic acids or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain) or the like. Examples of metabolic disorders include and are not limited to diabetes, insulin resistance, dyslipidemia, metabolic syndrome or the like.

The term “enhancing enteroendocrine peptide secretion” refers to a sufficient increase in the level of the enteroendocrine peptide agent to, for example, decrease hunger in a subject, to curb appetite in a subject and/or decrease the food intake of a subject or individual and/or treat any disease or disorder described herein. In some embodiments, enhanced enteroendocrine peptide secretion reverses or alleviates symptoms of congestive heart failure, ventricular dysfunction, toxic hypervolemia, polycystic ovary syndrome, inflammatory bowel disease, impaired bowel integrity, short bowel syndrome, gastritis, peptic ulcer, bile reflux, esophagitis, or irritable bowel syndrome.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-toluenenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

### Methods

Provided herein, in certain embodiments are methods for treating obesity and/or diabetes comprising administering a therapeutically effective amount of a bile acid sequestrant (e.g., a labile bile acid sequestrant) to an individual in need thereof. Also provided herein are methods of reducing blood plasma glucose levels of an individual comprising administering a therapeutically effective amount of a bile acid sequestrant (e.g., a labile bile acid sequestrant) to an individual in need thereof. Provided herein are methods for stimulating L-cells in the gastrointestinal tract of an individual comprising administering a therapeutically effective amount of a bile acid sequestrant (e.g., a labile bile acid sequestrant) to an individual in need thereof. In some embodiments of the methods described herein, an increase in concentration of bile acids in the vicinity of L-cell increases the secretion of enteroendocrine peptides, including GLP-1, PYY and/or oxyntmodulin from L-cells. In some instances a higher concentration of GLP-1 and/or PYY and/or oxynotmodulin in the blood and/or plasma of an individual increases insulin sensitivity of the individual and/or slows down gastric emptying and/or induces a feeling of satiety.

In certain embodiments, provided herein is a method of reducing food intake in an individual in need thereof comprising contacting the gastrointestinal tract of the individual with a therapeutically effective amount of a bile acid sequestrant (e.g., a labile bile acid sequestrant). In certain embodiments, provided herein is a method of reducing weight of an individual in need thereof comprising contacting the gastrointestinal tract of the individual with a therapeutically effective amount of a bile acid sequestrant (e.g., a labile bile acid sequestrant). In some embodiments, the method provides for inhibition of bile salt recycling upon administration of any of the compounds described herein to an individual. In some embodiments, a bile acid sequestrant (e.g., a labile bile acid sequestrant) described herein is administered to the individual orally, enterically or rectally. In specific embodiments, the bile acid sequestrant (e.g., a labile bile acid sequestrant) is administered orally with an optional enteric coating. In some embodiments, a bile acid sequestrant (e.g., a labile bile acid sequestrant) described herein is delivered to the small intestine of an individual whereupon it is charged with endogenous bile acids and transports these endogenous bile acids to the colon and/or rectum, where it releases at least a portion of the bile acids charged therein. In some embodiments, a bile acid sequestrant (e.g., a labile bile acid sequestrant) described herein increases the concentration of bile acids in the distal ileum, the colon and/or the rectum and induces secretion of enteroendocrine peptide products from the L-cells of the gastrointestinal tract. In certain instances administration of a therapeutically effective amount of a bile acid sequestrant (e.g., a labile bile acid sequestrant) described herein to an individual in need thereof increases the secretion of enteroendocrine peptide products (e.g., GLP-1, PYY, oxyntomodulin or the like) from L-cells that line the gastrointestinal tract. In some embodiments, elevated levels of GLP-1 slow down gastric emptying, and/or inhibit or reduce meal-stimulated gastric secretion, thereby reducing food intake in the individual. In some embodiments, a bile acid sequestrant (e.g., a labile bile acid sequestrant) described herein is administered in combination with a DPP-IV inhibitor. In some embodiments, the methods described herein are methods for reducing food intake in obese or morbidly overweight individuals. In some embodiments, a reduction in food intake reduces the weight of an individual (e.g., an obese or morbidly overweight individual)

In some embodiments, a method described herein provides for increased secretion of multiple enteroendocrine peptide products (e.g., GLP-1, PYY, oxyntomodulin, or the like) in the distal GI tract (e.g., colon and/or rectum). In certain instances, the increase of multiple enteroendocrine peptide products provides for an enhanced efficacy and/or enhanced duration of beneficial therapy. In some instances, therapy that involves administration and/or stimulation of a single enteroendocrine peptide product may result in a decrease in or elimination of efficacy over short periods of time.

In some embodiments, levels of GLP-1 in the blood and/or plasma of the individual are increased by about 1.1 times to about 30 times the level of GLP-1 in the blood and/or plasma of the individual prior to administration of the bile acid sequestrant. In certain embodiments, levels of GLP-1 in the blood and/or plasma of the individual are increased by about 1.5 times to about 20 times the level of GLP-1 in the blood and/or plasma of the individual prior to administration of the bile acid sequestrant. In some embodiments, levels of GLP-1 in the blood and/or plasma of the individual are increased by about 2 times to about 10 times the level of GLP-1 in the blood and/or plasma of the individual prior to administration of the bile acid sequestrant. In some embodiments, levels of GLP-1 in the blood and/or plasma of the individual are increased by about 2 times to about 6 times or about 2 times to about 10 times the level of GLP-1 in the blood and/or plasma of the individual prior to administration of the bile acid sequestrant. In some embodiments, levels of GLP-1 in the blood and/or plasma of the individual are increased by about 2 times to about 3 times the level of GLP-1 in the blood and/or plasma of the individual prior to administration of the bile acid sequestrant.

In certain embodiments, provided herein is a method for inducing satiety in an individual in need thereof comprising delivering to the gastrointestinal tract of the individual with a therapeutically effective amount of a bile acid sequestrant (e.g., a labile bile acid sequestrant). In some embodiments, the method provides for transport of bile acids from the small intestines to the colon and/or rectum of the individual. In some embodiments, a bile acid sequestrant (e.g., a labile bile acid sequestrant) described herein is administered to the individual orally, enterically or rectally. In some embodiments, a bile acid sequestrant (e.g., a labile bile acid sequestrant) described herein increases the concentration of bile acids in the distal ileum, the colon and/or the rectum and induces secretion of enteroendocrine peptide products from the L-cells of the gastrointestinal tract. In certain instances administration of a therapeutically effective amount of a bile acid sequestrant (e.g., a labile bile acid sequestrant) described herein to an individual in need thereof increases the secretion of enteroendocrine peptide products (e.g., GLP-1, PYY, oxyntomodulin or the like) from L-cells that line the gastrointestinal tract. In some embodiments, elevated levels of GLP-1 slow down gastric emptying, and induce a feeling of fullness in an individual. In some embodiments, a bile acid sequestrant (e.g., a labile bile acid sequestrant) described herein is administered in combination with a DPP-IV inhibitor. In some instances, inhibition of DPP-IV reduces the degradation of enteroendocrine peptide products (e.g. GLP-1) thereby prolonging the delay in gastric emptying and sustaining the feeling of satiety and/or fullness. In some embodiments of the methods, a bile acid sequestrant (e.g., a labile bile acid sequestrant) described herein is administered to a non-diabetic individual. In some embodiments, a bile acid sequestrant (e.g., a labile bile acid sequestrant) described herein is administered to an obese or morbidly overweight individual.

In certain embodiments, provided herein is a method for treating metabolic disorders in an individual in need thereof comprising contacting the gastrointestinal tract of the individual with a therapeutically effective amount of a bile acid sequestrant (e.g., a labile bile acid sequestrant). In some embodiments, a bile acid sequestrant (e.g., a labile bile acid sequestrant) described herein is administered to the individual orally, enterically or rectally. In some embodiments, a bile acid sequestrant (e.g., a labile bile acid sequestrant) described herein increases the concentration of bile acids in the distal ileum, the colon and/or the rectum and induces secretion of enteroendocrine peptide products from the L-cells of the gastrointestinal tract. In certain instances administration of a therapeutically effective amount of a bile acid sequestrant (e.g., a labile bile acid sequestrant) described herein to an individual in need thereof increases the secretion of enteroendocrine peptide products (e.g., GLP-1, PYY, oxyntomodulin or the like) from L-cells that line the gastrointestinal tract. In some embodiments, elevated levels of GLP-1 reduce glucose levels in blood. In some instances, elevated levels of GLP-1 increase insulin sensitivity in a hyperglycemic individual. In some embodiments, a bile acid sequestrant (e.g., a labile bile acid sequestrant) described herein is administered in combination with a DPP-IV inhibitor. In some instances, inhibition of DPP-IV reduces the degradation of enteroendocrine peptide products (e.g. GLP-1) thereby prolonging the effect of GLP-1 in reducing blood glucose levels.

In some embodiments, levels of glucose in the blood and/or plasma of the individual are reduced by at least 5% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 10% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 20% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 30% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 40% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 50% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 60% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 70% compared to the level of glucose in the blood and/or plasma of the individual prior to administration of the bile acid sequestrant (e.g., labile bile acid sequestrant). In some embodiments, levels of glucose in the blood and/or plasma of the individual are reduced by at least 80% compared to the level of glucose in the blood and/or plasma of the individual prior to administration of the bile acid sequestrant (e.g., labile bile acid sequestrant). In some embodiments, reduced blood and/or plasma glucose levels in the individual are maintained for at least 1 hours compared to blood and/or plasma glucose levels in the individual prior to administration of the bile acid sequestrant. In some embodiments, reduced blood and/or plasma glucose levels in the individual are maintained for at least 2 hours compared to blood and/or plasma glucose levels in the individual prior to administration of the bile acid sequestrant. In some embodiments, reduced blood and/or plasma glucose levels in the individual are maintained for at least 4 hours compared to blood and/or plasma glucose levels in the individual prior to administration of the bile acid sequestrant. In some embodiments, reduced blood and/or plasma glucose levels in the individual are maintained for at least 6 hours compared to blood and/or plasma glucose levels in the individual prior to administration of the bile acid sequestrant. In some embodiments, reduced blood and/or plasma glucose levels in the individual are maintained for at least 8 hours compared to blood and/or plasma glucose levels in the individual prior to administration of the bile acid sequestrant. In some embodiments, reduced blood and/or plasma glucose levels in the individual are maintained for at least 12 hours compared to blood and/or plasma glucose levels in the individual prior to administration of the bile acid sequestrant. In some embodiments, reduced blood and/or plasma glucose levels in the individual are maintained for at least 24 hours compared to blood and/or plasma glucose levels in the individual prior to administration of the bile acid sequestrant.

In some embodiments, administration of a bile acid sequestrant (e.g., a labile bile acid sequestrant) inhibitor described herein allows for treatment of a metabolic disorder without the side effects associated with conventional therapies (e.g., biguanides such as metformin, DDPIV inhibitors or the like) for metabolic disorders.

In some embodiments, provided herein is a method of treating a metabolic disease or disorder by administering to an individual in need thereof a therapeutically effective amount of a bile acid sequestrant (e.g., a labile bile acid sequestrant) described herein in combination with a reduced dose of a biguanide (e.g., metformin) or a DPP-IV inhibitor (e.g., sitagliptin) or a TGR5 agonist, or a GPR119 agonist, or a GPR120 agonist, or a GPR40 agonist, or a GPR43 agonist, or a GPR154 agonist.

In some embodiments of any of the methods described herein, administration of a bile acid sequestrant (e.g., a labile bile acid sequestrant) described herein increases the level of GLP-1 in the blood and/or plasma of an individual by from about 1.5 times to about 10 times compared to the level of GLP-1 in the blood and/or plasma of the individual prior to administration of the bile acid sequestrant (e.g., labile bile acid sequestrant). In some embodiments of any of the methods described herein, administration of a bile acid sequestrant (e.g., a labile bile acid sequestrant) described herein increases the level of GLP-1 in the blood and/or plasma of an individual by from about 2 times to about 8 times compared to the level of GLP-1 in the blood and/or plasma of the individual prior to administration of a bile acid sequestrant (e.g., a labile bile acid sequestrant). In some embodiments of any of the methods described herein, administration of a bile acid sequestrant (e.g., a labile bile acid sequestrant) described herein increases the level of GLP-1 in the blood and/or plasma of an individual by from about 2 times to about 6 times compared to the level of GLP-1 in the blood and/or plasma of the individual prior to administration of a bile acid sequestrant (e.g., a labile bile acid sequestrant). In some instances, an increase in GLP-1 level of from about 2 times to about 3 times following the administration of a bile acid sequestrant (e.g., a labile bile acid sequestrant) described herein compared to the level of GLP-1 in the blood and/or plasma of the individual prior to administration of a bile acid sequestrant (e.g., a labile bile acid sequestrant) is associated with an anti-diabetic effect. In some instances, an increase in GLP-1 level of from about 3 times to about 8 times following the administration of a bile acid sequestrant (e.g., a labile bile acid sequestrant) described herein compared to the level of GLP-1 in the blood and/or plasma of the individual prior to administration of a bile acid sequestrant (e.g., a labile bile acid sequestrant) is associated with reduction in food intake and/or induction of satiety and/or weight loss.

In certain embodiments of any of the methods described herein, administration of a bile acid sequestrant (e.g., a labile bile acid sequestrant) reduces blood and/or plasma sugar levels by at least 20%, at least 30%, at least 40%, at least 50% at least 60%, at least 70% or at least 80% compared to blood and/or plasma sugar levels prior to administration of a bile acid sequestrant (e.g., a labile bile acid sequestrant). In some embodiments of any of the methods described herein, administration of a bile acid sequestrant (e.g., a labile bile acid sequestrant) reduces blood and/or plasma sugar levels by at least 20% compared to blood and/or plasma sugar levels prior to administration of a bile acid sequestrant (e.g., a labile bile acid sequestrant). In some embodiments of any of the methods described herein, administration of a bile acid sequestrant (e.g., a labile bile acid sequestrant) reduces blood and/or plasma sugar levels by at least 30% compared to blood and/or plasma sugar levels prior to administration of a bile acid sequestrant (e.g., a labile bile acid sequestrant). In some embodiments of any of the methods described herein, administration of a bile acid sequestrant (e.g., a labile bile acid sequestrant) reduces blood and/or plasma sugar levels by at least 40% compared to blood and/or plasma sugar levels prior to administration of a bile acid sequestrant (e.g., a labile bile acid sequestrant).

In some embodiments of any of the methods described herein, administration of a bile acid sequestrant (e.g., a labile bile acid sequestrant) reduces blood and/or plasma sugar levels for a longer period of time (e.g., at least 24 hours) compared to reduction in blood and/or plasma sugar levels upon administration of metformin. In some embodiments of any of the methods described herein, administration of a single dose of a bile acid sequestrant (e.g., a labile bile acid sequestrant) sustains reduced blood and/or plasma sugar levels for at least 6 hours, at least 12 hours, at least 14 hours, at least 16 hours, at least 18 hours, at least 20 hours, at least 24 hours, at least 30 hours, at least 36 hours or at least 48 hours compared to reduction in blood and/or plasma sugar levels upon administration of a single dose of metformin.

In some embodiments of any of the methods described herein, administration of a bile acid sequestrant (e.g., a labile bile acid sequestrant) results in higher levels of GLP-1 in blood and/or plasma of an individual compared to levels of GLP-1 in blood and/or plasma of a normal individual. In some embodiments of any of the methods described herein, administration of a bile acid sequestrant (e.g., a labile bile acid sequestrant) results in higher levels of GLP-1 in blood and/or plasma of an individual compared to levels of GLP-1 in blood and/or plasma of an individual that has been administered a DPP-IV inhibitor.

Also provided herein is a method for treating conditions that are ameliorated by increased secretion of L-cell enteroendocrine peptides by contacting the gastrointestinal tract of an individual in need thereof with a therapeutically effective amount of any compound described herein. L-cells are highly specialized gut enteroendocrine cells expressed along the gastrointestinal tract. The majority of L cells are located in the distal gastrointestinal tract, predominantly the ileum and colon. The cells in the enteric endocrine system do not secrete their hormone continuously. Instead, they respond to changes in the environment within the lumen of the digestive tube, including changes in bile acid concentrations in the lumen of the digestive tube. The apical border of L-cells is in contact with the contents of the gastrointestinal lumen. Enteroendocrine peptides secreted by L-cells include GLP-1, GLP-2, PYY and oxyntomodulin. In certain instances, the methods described herein enhance L-cell secretion of GLP-1, GLP-2, PYY or oxyntomodulin or combinations thereof. In certain instances, delivery of the bile acid sequestrant to the GI tract increases the concentration of bile acids in the vicinity of L-cells thereby enhancing the release of enteroendocrine peptides.

Conditions that are mediated by L-cell enteroendocrine peptides include congestive heart failure, ventricular dysfunction, toxic hypervolemia, polycystic ovary syndrome, inflamatory bowel disease, impaired bowel integrity, short bowel syndrome, gastritis, peptic ulcer, irritable bowel disease or the like.

Administration of a compound described herein is achieved in any suitable manner including, by way of non-limiting example, by oral, enteric, parenteral (e.g., intravenous, subcutaneous, intramuscular), intranasal, buccal, topical, rectal, or transdermal administration routes.

In certain embodiments, a compound or a composition comprising a compound described herein is administered for prophylactic and/or therapeutic treatments. In therapeutic applications, the compositions are administered to an individual already suffering from a disease or condition, in an amount sufficient to cure or at least partially arrest the symptoms of the disease or condition. In various instances, amounts effective for this use depend on the severity and course of the disease or condition, previous therapy, the individual's health status, weight, and response to the drugs, and the judgment of the treating physician.

In prophylactic applications, compounds or compositions containing compounds described herein are administered to an individual susceptible to or otherwise at risk of a particular disease, disorder or condition. In certain embodiments of this use, the precise amounts of compound administered depend on the individual's state of health, weight, and the like. Furthermore, in some instances, when a compound or composition described herein is administered to an individual, effective amounts for this use depend on the severity and course of the disease, disorder or condition, previous therapy, the individual's health status and response to the drugs, and the judgment of the treating physician.

In certain instances, wherein following administration of a selected dose of a compound or composition described herein, an individual's condition does not improve, upon the doctor's discretion the administration of a compound or composition described herein is optionally administered chronically, that is, for an extended period of time, including throughout the duration of the individual's life in order to ameliorate or otherwise control or limit the symptoms of the individual's disorder, disease or condition.

In certain embodiments, an effective amount of a given agent varies depending upon one or more of a number of factors such as the particular compound, disease or condition and its severity, the identity (e.g., weight) of the subject or host in need of treatment, and is determined according to the particular circumstances surrounding the case, including, e.g., the specific agent being administered, the route of administration, the condition being treated, and the subject or host being treated. In some embodiments, doses administered include those up to the maximum tolerable dose. In certain embodiments, about 0.001-5000 mg per day, from about 0.001-1500 mg per day, about 0.001 to about 100 mg/day, about 0.001 to about 50 mg/day, or about 0.001 to about 30 mg/day, or about 0.001 to about 10 mg/day of a compound described herein is administered. In various embodiments, the desired dose is conveniently presented in a single dose or in divided doses administered simultaneously (or over a short period of time) or at appropriate intervals, for example as two, three, four or more sub-doses per day. In various embodiments, a single dose is from about 0.001 mg/kg to about 500 mg/kg. In various embodiments, a single dose is from about 0.001, 0.01, 0.1, 1, or 10 mg/kg to about 10, 50, 100, or 250 mg/kg. In various embodiments, a single dose of a bile acid sequestrant (e.g., a labile bile acid sequestrant) is from about 0.001 mg/kg to about 100 mg/kg. In various embodiments, a single dose of a bile acid sequestrant (e.g., a labile bile acid sequestrant) is from about 0.001 mg/kg to about 50 mg/kg. In various embodiments, a single dose of a bile acid sequestrant (e.g., a labile bile acid sequestrant) is from about 0.001 mg/kg to about 10 mg/kg. In various embodiments, a single dose of a bile acid sequestrant (e.g., a labile bile acid sequestrant) is administered every 6 hours, every 12 hours, every 24 hours, every 48 hours, every 72 hours, every 96 hours, every 5 days, every 6 days, or once a week.

In some embodiments, a composition comprises a method described herein comprises administering an effective amount of a bile acid sequestrant, wherein the effective amount of bile acid sequestrant is an amount sufficient to provide a bile acid concentration in the colon of greater than 3 mM. In certain embodiments, the effective amount of bile acid sequestrant is an amount sufficient to provide a bile acid concentration in the colon of greater than 4 mM. In some embodiments, the effective amount of bile acid sequestrant is an amount sufficient to provide a bile acid concentration in the colon of greater than 5 mM. In certain embodiments, the effective amount of bile acid sequestrant is an amount sufficient to provide a bile acid concentration in the colon of greater than 6 mM. In some embodiments, the effective amount of bile acid sequestrant is an amount sufficient to provide a bile acid concentration in the colon of greater than 8 mM. In certain embodiments, the effective amount of bile acid sequestrant is an amount sufficient to provide a bile acid concentration in the colon of greater than 10 mM.

In some embodiments, a composition comprises a method described herein comprises administering an effective amount of a bile acid sequestrant, wherein the effective amount of bile acid sequestrant is an amount sufficient to provide a bile acid concentration increase in the colon of greater than 5% w/w. In certain embodiments, the effective amount of bile acid sequestrant is an amount sufficient to provide a bile acid concentration increase in the colon of greater than 10% w/w. In certain embodiments, the effective amount of bile acid sequestrant is an amount sufficient to provide a bile acid concentration increase in the colon of greater than 20% w/w. In certain embodiments, the effective amount of bile acid sequestrant is an amount sufficient to provide a bile acid concentration increase in the colon of greater than 25% w/w. In certain embodiments, the effective amount of bile acid sequestrant is an amount sufficient to provide a bile acid concentration increase in the colon of greater than 30% w/w. In certain embodiments, the effective amount of bile acid sequestrant is an amount sufficient to provide a bile acid concentration increase in the colon of greater than 50% w/w. In certain embodiments, the effective amount of bile acid sequestrant is an amount sufficient to provide a bile acid concentration increase in the colon of greater than 75% w/w. In certain embodiments, the effective amount of bile acid sequestrant is an amount sufficient to provide a bile acid concentration increase in the colon of greater than 100% w/w. In certain embodiments, the effective amount of bile acid sequestrant is an amount sufficient to provide a bile acid concentration increase in the colon of greater than 150% w/w. In certain embodiments, the effective amount of bile acid sequestrant is an amount sufficient to provide a bile acid concentration increase in the colon of greater than 200% w/w.

In certain instances, there are a large number of variables in regard to an individual treatment regime, and considerable excursions from these recommended values are considered within the scope described herein. Dosages described herein are optionally altered depending on a number of variables such as, by way of non-limiting example, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are optionally determined by pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. In certain embodiments, data obtained from cell culture assays and animal studies are used in formulating a range of dosage for use in human. In specific embodiments, the dosage of compounds described herein lies within a range of circulating concentrations that include the ED₅₀ with minimal toxicity. The dosage optionally varies within this range depending upon the dosage form employed and the route of administration utilized.

In some embodiments, the bile acid sequestrant is administered before ingestion of food or with food. In certain embodiments, the bile acid sequestrant is administered to an individual in the fed state. In other embodiments, the bile acid sequestrant is administered in the fasted state. In specific embodiments, the bile acid sequestrant is administered less than about 60 minutes before ingestion of food. In more specific embodiments, bile acid sequestrant is administered less than about 30 minutes before ingestion of food. In still more specific embodiments, bile acid sequestrant is administered less than about 15 minutes before ingestion of food. In yet more specific embodiments, bile acid sequestrant is administered less than about 5 minutes before ingestion of food.

In certain embodiments, the bile acid sequestrants (e.g., labile bile acid sequestrants), compositions or methods described herein are non-systemic. In some embodiments, administration of a bile acid sequestrant described herein does not provide for systemic delivery of the bile acid sequestrant (e.g., a substantial portion of the bile acid sequestrant is not systemically absorbed). In certain embodiments, non-systemic compositions described herein deliver less than 25% w/w of the bile acid sequestrant systemically. In certain embodiments, non-systemic compositions described herein deliver less than 20% w/w of the bile acid sequestrant systemically. In certain embodiments, non-systemic compositions described herein deliver less than 15% w/w of the bile acid sequestrant systemically. In certain embodiments, non-systemic compositions described herein deliver less than 10% w/w of the bile acid sequestrant systemically. In certain embodiments, non-systemic compositions described herein deliver less than 5% w/w of the bile acid sequestrant systemically. In certain embodiments, non-systemic compositions described herein deliver less than 4% w/w of the bile acid sequestrant systemically. In certain embodiments, non-systemic compositions described herein deliver less than 3% w/w of the bile acid sequestrant systemically. In certain embodiments, non-systemic compositions described herein deliver less than 2% w/w of the bile acid sequestrant systemically. In certain embodiments, non-systemic compositions described herein deliver less than 1% w/w of the bile acid sequestrant systemically. In some embodiments, systemic absorption is determined in any suitable manner, including the total circulating amount, the amount cleared after administration, or the like.

### Oral Administration for Colonic Delivery

In certain aspects, the composition or formulation containing one or more labile bile sequestrant linked to an exogenous bile acid, a bile salt, a bile acid mimic, or a bile salt mimic, free fatty acids, or a free fatty acids mimic is orally administered for local delivery to the colon and/or rectum. Unit dosage forms of such compositions include a pill, tablet or capsules formulated for enteric delivery to colon. In certain embodiments, such pills, tablets or capsule contain the compositions described herein entrapped or embedded in microspheres. In some embodiments, microspheres include, by way of non-limiting example, chitosan microcores HPMC capsules and cellulose acetate butyrate (CAB) microspheres. In certain embodiments, oral dosage forms are prepared using conventional methods known to those in the field of pharmaceutical formulation. For example, in certain embodiments, tablets are manufactured using standard tablet processing procedures and equipment. An exemplary method for forming tablets is by direct compression of a powdered, crystalline or granular composition containing the active agent(s), alone or in combination with one or more carriers, additives, or the like. In alternative embodiments, tablets are prepared using wet-granulation or dry-granulation processes. In some embodiments, tablets are molded rather than compressed, starting with a moist or otherwise tractable material.

In certain embodiments, a composition described herein comprises one or more labile bile sequestrant linked to an exogenous bile acid, a bile salt, a bile acid mimic, or a bile salt mimic, free fatty acids, or a free fatty acids mimic in association with a matrix (e.g., a matrix comprising hypermellose) that allows for controlled release of an active agent in the colon or the rectum. In some embodiments, a composition comprises a polymer that is pH sensitive (e.g., a MMX™ matrix from Cosmo Pharmaceuticals) and allows for controlled release of an active agent in the colon or the rectum. Examples of such pH sensitive polymers suitable for controlled release include and are not limited to polyacrylic polymers (e.g., anionic polymers of methacrylic acid and/or methacrylic acid esters, e.g., Carbopol® polymers) that comprise acidic groups (e.g., —COOH, —SO₃H) and swell in basic pH of the intestine (e.g., pH of about 7 to about 8). In some embodiments, a composition suitable for controlled release in the colon or the rectum comprises microparticulate active agent (e.g., micronized active agent). In some embodiments, a non-enzymatically degrading poly(dl-lactide-co-glycolide) (PLGA) core is suitable for delivery to the colon or the rectum. In some embodiments, a dosage form is coated with an enteric polymer (e.g., Eudragit® S-100, cellulose acetate phthalate, polyvinylacetate phthalate, hydroxypropylmethylcellulose phthalate, anionic polymers of methacrylic acid, methacrylic acid esters or the like) for site specific delivery to the colon or the rectum.

In some embodiments, bacterially activated systems are suitable for targeted delivery to the colon or the rectum. In some embodiments, the enzyme is a bacterial enzyme found in high concentration in human colon or rectum relative to the concentration found in the small intestine. Examples of micro-flora activated systems include dosage forms comprising pectin, galactomannan, and/or Azo hydrogels and/or glycoside conjugates (e.g., conjugates of D-galactoside, β-D-xylopyranoside or the like) of the active agent. Examples of gastrointestinal micro-flora enzymes include bacterial glycosidases such as, for example, D-galactosidase, β-D-glucosidase, α-L-arabinofuranosidase, β-D-xylopyranosidase or the like. In some embodiments, the labile bile acid sequestrant is a time dependent bile acid sequestrant (i.e., the bile acid sequesters the bile acid and/or salts thereof and after a time releases at least a portion of the bile acid and/or salts thereof). In some embodiments, a time dependent bile acid sequestrant is an agent that degrades in an aqueous environment over time. In certain embodiments, a labile bile acid sequestrant described herein is a bile acid sequestrant that has a low affinity for bile acid and/or salts thereof, thereby allowing the bile acid sequestrant to continue to sequester bile acid and/or salts thereof in an environment where the bile acids and/or salts thereof are present in high concentration and release them in an environment wherein bile acids and/or salts thereof are present in a lower relative concentration. In some embodiments, the labile bile acid sequestrant has a high affinity for a primary bile acid and a low affinity for a secondary bile acid, allowing the bile acid sequestrant to sequester a primary bile acid or salt thereof and subsequently release a secondary bile acid or salt thereof as the primary bile acid or salt thereof is converted (e.g., metabolized) to the secondary bile acid or salt thereof. In some embodiments, the labile bile acid sequestrant is a pH dependent bile acid sequestrant. In some embodiments, the pH dependent bile acid sequestrant has a high affinity for bile acid at a pH of 7 or below and a low affinity for bile acid at a pH above 7. In certain embodiments, the pH dependent bile acid sequestrant degrades at a pH above 7.

In certain embodiments, tablets prepared for oral administration contain various excipients, including, by way of non-limiting example, binders, diluents, lubricants, disintegrants, fillers, stabilizers, surfactants, preservatives, coloring agents, flavoring agents and the like. In some embodiments, binders are used to impart cohesive qualities to a tablet, ensuring that the tablet remains intact after compression. Suitable binder materials include, by way of non-limiting example, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), Veegum, and combinations thereof. In certain embodiments, diluents are utilized to increase the bulk of the tablet so that a practical size tablet is provided. Suitable diluents include, by way of non-limiting example, dicalcium phosphate, calcium sulfate, lactose, cellulose, kaolin, mannitol, sodium chloride, dry starch, powdered sugar and combinations thereof. In certain embodiments, lubricants are used to facilitate tablet manufacture; examples of suitable lubricants include, by way of non-limiting example, vegetable oils such as peanut oil, cottonseed oil, sesame oil, olive oil, corn oil, and oil of theobroma, glycerin, magnesium stearate, calcium stearate, stearic acid and combinations thereof. In some embodiments, disintegrants are used to facilitate disintegration of the tablet, and include, by way of non-limiting example, starches, clays, celluloses, algins, gums, crosslinked polymers and combinations thereof. Fillers include, by way of non-limiting example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. In certain embodiments, stabilizers are used to inhibit or retard drug decomposition reactions that include, by way of example, oxidative reactions. In certain embodiments, surfactants are anionic, cationic, amphoteric or nonionic surface active agents.

Covalent Linkage of the Drug with a Carrier

In some embodiments, strategies used for colon targeted delivery include, by way of non-limiting example, covalent linkage of one or more labile bile sequestrant linked to an exogenous bile acid, a bile salt, a bile acid mimic, or a bile salt mimic, free fatty acids, or a free fatty acids mimic, coating the dosage form with a pH-sensitive polymer for delivery upon reaching the pH environment of the colon, using redox sensitive polymers, using a time released formulation, utilizing coatings that are specifically degraded by colonic bacteria, using bioadhesive system and using osmotically controlled drug delivery systems.

In certain embodiments of such oral administration of a composition containing one or more labile bile sequestrant described herein involves covalent linking a labile bile sequestrant linked to an exogenous bile acid, a bile salt, a bile acid mimic, or a bile salt mimic, free fatty acids, or a free fatty acids mimic wherein upon oral administration the linked moiety remains intact in the stomach and small intestine. Upon entering the colon the covalent linkage is broken by the change in pH, enzymes, and/or degradation by intestinal microflora. In certain embodiments, the covalent linkage between a labile bile sequestrant and an exogenous bile acid, a bile salt, a bile acid mimic, or a bile salt mimic, free fatty acids, or a free fatty acids mimic includes, by way of non-limiting example, azo linkage, glycoside conjugates, glucuronide conjugates, cyclodextrin conjugates, dextran conjugates, and amino-acid conjugates (high hydrophilicity and long chain length of the carrier amino acid).

Coating with Polymers: pH-Sensitive Polymers

In some embodiments, the oral dosage forms described herein are coated with an enteric coating to facilitate the delivery of one or more labile bile sequestrant to the colon and/or rectum. In certain embodiments, an enteric coating is one that remains intact in the low pH environment of the stomach, but readily dissolved when the optimum dissolution pH of the particular coating is reached which depends upon the chemical composition of the enteric coating. The thickness of the coating will depend upon the solubility characteristics of the coating material. In certain embodiments, the coating thicknesses used in such formulations described herein range from about 25 μm to about 200 μm.

In certain embodiments, the compositions or formulations described herein are coated such that one or more labile bile sequestrant of the composition or formulation is delivered to the colon and/or rectum without absorbing at the upper part of the intestine. In a specific embodiment, specific delivery to the colon and/or rectum is achieved by coating of the dosage form with polymers that degrade only in the pH environment of the colon. In alternative embodiments, the composition is coated with an enteric coat that dissolves in the pH of the intestines and an outer layer matrix that slowly erodes in the intestine. In some of such embodiments, the matrix slowly erodes until only a core composition comprising one or more labile bile sequestrant (and, in some embodiments, linked to an exogenous bile acid, a bile salt, a bile acid mimic, or a bile salt mimic, free fatty acids, or a free fatty acids mimic) is left and the core is delivered to the colon and/or rectum.

In certain embodiments, pH-dependent systems exploit the progressively increasing pH along the human gastrointestinal tract (GIT) from the stomach (pH 1-2 which increases to 4 during digestion), small intestine (pH 6-7) at the site of digestion and it to 7-8 in the colon or the rectum. In certain embodiments, dosage forms for oral administration of the compositions described herein are coated with pH-sensitive polymer(s) to provide delayed release and protect the enteroendocrine peptide secretion enhancing agents from gastric fluid. In certain embodiments, such polymers are be able to withstand the lower pH values of the stomach and of the proximal part of the small intestine, but disintegrate at the neutral or slightly alkaline pH of the terminal ileum and/or ileocecal junction. Thus, in certain embodiments, provided herein is an oral dosage form comprising a coating, the coating comprising a pH-sensitive polymer. In some embodiments, the polymers used for colon and/or rectum targeting include, by way of non-limiting example, methacrylic acid copolymers, methacrylic acid and methyl methacrylate copolymers, Eudragit L100, Eudragit S100, Eudragit L-30D, Eudragit FS-30D, Eudragit L100-55, polyvinylacetate phthalate, hyrdoxypropyl ethyl cellulose phthalate, hyrdoxypropyl methyl cellulose phthalate 50, hyrdoxypropyl methyl cellulose phthalate 55, cellulose acetate trimelliate, cellulose acetate phthalate and combinations thereof.

In certain embodiments, oral dosage forms suitable for delivery to the colon and/or rectum comprise a coating that has a biodegradable and/or bacteria degradable polymer or polymers that are degraded by the microflora (bacteria) in the colon. In such biodegradable systems suitable polymers include, by way of non-limiting example, azo polymers, linear-type-segmented polyurethanes containing azo groups, polygalactomannans, pectin, glutaraldehyde crosslinked dextran, polysaccharides, amylose, guar gum, pectin, chitosan, inulin, cyclodextrins, chondroitin sulphate, dextrans, locust bean gum, chondroitin sulphate, chitosan, poly(-caprolactone), polylactic acid and poly(lactic-co-glycolic acid).

In certain embodiments of such oral administration of compositions containing one or more enteroendocrine peptide secretion enhancing agents described herein, the compositions are delivered to the colon without absorbing at the upper part of the intestine by coating of the dosage forms with redox sensitive polymers that are degraded by the microflora (bacteria) in the colon. In such biodegradable systems such polymers include, by way of non-limiting example, redox-sensitive polymers containing an azo and/or a disulfide linkage in the backbone.

In some embodiments, compositions formulated for delivery to the colon and/or rectum are formulated for time-release. In some embodiments, time release formulations resist the acidic environment of the stomach, thereby delaying the release of the enteroendocrine peptide secretion enhancing agents until the dosage form enters the colon and/or rectum.

In certain embodiments the time released formulations described herein comprise a capsule with hydrogel plug. In certain embodiments, the capsule and hydrogel plug are covered by a water-soluble cap and the whole unit is coated with an enteric polymer. When the capsule enters the small intestine the enteric coating dissolves and the hydrogels plug swells and dislodges from the capsule after a period of time and the composition is released from the capsule. The amount of hydrogel is used to adjust the period of time to the release the contents.

In some embodiments, provided herein is an oral dosage form comprising a multi-layered coat, wherein the coat comprises different layers of polymers having different pH-sensitivities. As the coated dosage form moves along GIT the different layers dissolve depending on the pH encountered. Polymers used in such formulations include, by way of non-limiting example, polymethacrylates with appropriate pH dissolution characteristics, Eudragit® RL and Eudragit®RS (inner layer), and Eudragit® FS (outer layer). In other embodiments the dosage form is an enteric coated tablets having an outer shell of hydroxypropylcellulose or hydroxypropylmethylcellulose acetate succinate (HPMCAS).

In some embodiments, provided herein is an oral dosage form that comprises coat with cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and combinations thereof.

### Combinations

In some embodiments, the methods described herein comprise administration of a bile acid sequestrant (e.g., a labile bile acid sequestrant) and an additional therapeutic agent. In some embodiments, a second dosage form comprising an additional therapeutic agent (alternatively, the agents may be administered in a single dosage form) is administered. In certain embodiments, combination therapies the compositions described herein are administered as part of a treatment regimen. Therefore, additional therapeutic agents and/or additional pharmaceutical dosage form can be applied to a patient either directly or indirectly, and concomitantly or sequentially, with the compositions and formulations described herein.

In certain instances, provided herein are combination compositions and/or therapies comprising any bile acid sequestrant (e.g., labile bile acid sequestrant) described herein and an additional therapeutic agent. In some embodiments, the additional therapeutic agent is a L-cell endocrine peptide enhancer. In some instances, the L-cell endocrine peptide enhancer is a GLP-1 enhancer. In some embodiments, the GLP-1 enhancer is GLP-1, a GLP-1 secretion enhancer, a GLP-1 degradation inhibitor, the like, or a combination thereof. In certain instances, enhanced GLP-1 concentration provides a reduction in food intake and/or a reduction in gastric emptying in human subjects.

In some embodiments, the L-cell endocrine peptide enhancer is a GLP-2 enhancer. In certain instances, the GLP-2 enhancer is GLP-2, a GLP-2 secretion enhancer, a GLP-2 degradation inhibitor, the like, or a combination thereof. In certain instances, enhanced GLP-2 secretion inhibits gastric emptying and reduces intestinal permeability. In some instances, enhanced GLP-2 secretion inhibits gastric acid secretion.

In some instances, the L-cell endocrine peptide enhancer is a PYY enhancer. In some instances, enhanced secretion of PYY provides a reduction in sensation of hunger. In some instances, the L-cell endocrine peptide enhancer is a oxyntomodulin enhancer. In some instances, the enhanced secretion of oxyntomodulin inhibits meal-stimulated gastric secretion.

In some embodiments, provided herein are bile acid sequestrants (e.g., labile bile acid sequestrants) in combination with at least one additional agent. In some embodiments, provided herein is a composition or a combination treatment wherein the bile acid sequestrant (e.g., labile bile acid sequestrant) is combined with an Apical Sodium-dependent Bile Transporter (ASBT) inhibitor (e.g., an topically active ASBT inhibitor), a DPP-IV inhibitor, a TGR5 agonist, a GPR119 agonist, a GPR120 agonist, a GPR40 agonist, a GPR43 agonist, a GPR154 agonist, or the like, or a combination thereof. In various embodiments, any ASBTI or DPPIV inhibitor is optionally utilized. In some embodiments, provided herein are methods and compositions comprising a bile acid sequestrant (e.g., a labile bile acid sequestrant) and a second agent selected from an ASBT inhibitor, metformin, an incretin mimetic, a DPP-IV inhibitor, a TGR5 agonist, a GPR119 agonist, a GPR120 agonist, a GPR40 agonist, a GPR43 agonist, and a GPR154 agonist.

In some embodiments, the ASBTI is a compound of Formula I:

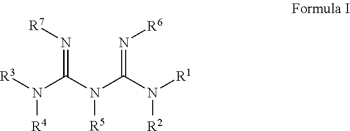

wherein R¹, R², R³, R⁴, R⁵, R⁶ and R⁷ are as described above.

In some embodiments, at least one of R¹, R², R³, R⁴, R⁵, R⁶ and R⁷ is H. In certain embodiments, R⁵, R⁶, R⁷ are H and R¹, R², R³ and R⁴ are alkyl, aryl, alkyl-aryl, or heteroalkyl. In some embodiments, R¹ and R² are H. In some embodiments, R¹, R², R⁵, R⁶ and R⁷ are H. In some embodiments, R⁶ and R⁷ together form a bond. In certain embodiments, R⁵, R⁶ and R⁷ are H, alkyl or O-alkyl.

In some embodiments, R¹ and R³ are -L-K. In some embodiments, R¹, R² and R³ are -L-K. In some embodiments, R³ and R⁴ are -L-K. In some embodiments, R¹ and R² together with the nitrogen to which they are attached form a 3-8 membered ring and the ring is substituted with -L-K. In some embodiments, R¹ or R² or R³ or R⁴ are aryl optionally substituted with -L-K. In some embodiments, R¹ or R² or R³ or R⁴ are alkyl optionally substituted with -L-K. In some embodiments, R¹ or R² or R³ or R⁴ are alky-aryl optionally substituted with -L-K. In some embodiments, R¹ or R² or R³ or R⁴ are heteroalkyl optionally substituted with -L-K.

In some embodiments, L is a C₁-C₇alkyl. In some embodiments, L is heteroalkyl. In certain embodiments, L is C₁-C₇alkyl-aryl. In some embodiments, L is C₁-C₇alkyl-aryl-C₁-C₇alkyl.

In certain embodiments, K is a non-protic charged group. In some specific embodiments, each K is a ammonium group. In some embodiments, each K is a cyclic non-protic ammonium group. In some embodiments, each K is an acyclic non-protic ammonium group.

In certain embodiments, each K is a cyclic non-protic ammonium group of structure:

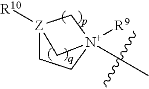

In certain embodiments, K is an acyclic non-protic ammonium group of structure:

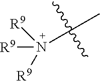

wherein p, q, R⁹, R¹⁰ and Z are as defined above. In certain embodiments, p is 1. In other embodiments, p is 2. In further embodiments, p is 3. In some embodiments, q is 0. In other embodiments, q is 1. In some other embodiments, q is 2.

The compounds further comprise 1, 2, 3 or 4 anionic counterions selected from Cl⁻, Br⁻, I⁻, R¹¹SO₃⁻, (SO₃⁻—R¹¹—SO₃⁻), R¹¹CO₂⁻, (CO₂⁻—R¹¹—CO₂⁻), (R¹¹)₂(P═O)O⁻ and (R¹¹)(P═O)O₂²⁻ wherein R¹¹ is as defined above. In some embodiments, the counterion is Cl⁻, Br⁻, I⁻, CH₂CO₂⁻, CH₃SO₃, or C₆H₅SO₃ or CO₂— (CH₂)₂—CO₂⁻. In some embodiments, the compound of Formula I has one K group and one counterion. In other embodiments, the compound of Formula I has one K group, and two molecules of the compound of Formula I have one counterion. In yet other embodiments, the compound of Formula I has two K groups and two counterions. In some other embodiments, the compound of Formula I has one K group comprising two ammonium groups and two counterions.

Other ASBT inhibitors are found in U.S. Patent Application Nos. 61/118,356, 61/239,657, and 61/239,663, which are hereby incorporated by reference for such disclosure. In other embodiments, compounds that inhibit ASBT or any recuperative bile acid transporters are compounds that are described in EP1810689, U.S. Pat. Nos. 6,458,851, 7,413,536, US Appl. Publication Nos. 2002/0147184, 2003/0119809, 2003/0149010, 2004/0014806, 2004/0092500, 2004/0180861, 2004/0180860, 2005/0031651, 2006/0069080, 2006/0199797, 2006/0241121, 2007/0065428, 2007/0066644, 2007/0161578, 2007/0197628, 2007/0203183, 2007/0254952, 2008/0070888, 2008/0070892, 2008/0070889, 2008/0070984, 2008/0089858, 2008/0096921, 2008/0161400, 2008/0167356, 2008/0194598, 2008/0255202, 2008/0261990, WO2002/50027, WO2005/046797, WO2006/017257, WO2006/105913, WO2006/105912, WO2006/116499, WO2006/117076, WO2006/121861, WO2006/122186, WO2006/124713, WO2007/050628, WO2007/101531, WO2007/134862, WO2007/140934, WO2007/140894, WO2008/028590, WO2008/033431, WO2008/033464, WO2008/031501, WO2008/031500, WO2008/033465, WO2008/034534, WO2008/039829, WO2008/064788, WO2008/064789, WO2008/088836, WO2008/104306, WO2008/124505, and WO2008/130616; the compounds described therein that inhibit recuperative bile acid transport are hereby incorporated herein by reference.

In certain embodiments, compounds that inhibit ASBT or any recuperative bile acid transporters are compounds described in WO93/16055, WO94/18183, WO94/18184, WO96/05188, WO96/08484, WO96/16051, WO97/33882, WO98/38182, WO99/35135, WO98/40375, WO99/64409, WO99/64410, WO00/01687, WO00/47568, WO00/61568, DE 19825804, WO00/38725, WO00/38726, WO00/38727 (including those compounds with a 2,3,4,5-tetrahydro-1-benzothiepine 1,1-dioxide structure), WO00/38728, WO01/66533, WO02/50051, EP0864582 (e.g. (3R,5R)-3-butyl-3-ethyl-1,1-dioxido-5-Phenyl-2,3,4,5-tetrahydro-1,4-benzo-thiazepin-8-yl(β-D-glucopyranosiduronic acid, WO94/24087, WO98/07749, WO98/56757, WO99/32478, WO99/35135, WO00/20392, WO00/20393, WO00/20410, WO00/20437, WO01/34570, WO00/35889, WO01/68637, WO01/68096, WO02/08211, WO03/020710, WO03/022825, WO03/022830, WO03/0222861, JP10072371, U.S. Pat. Nos. 5,910,494; 5,723,458; 5,817,652; 5,663,165; 5,998,400; 6,465,451, 5,994,391; 6,107,494; 6,387,924; 6,784,201; 6,875,877; 6,740,663; 6,852,753; 5,070,103, 6,114,322, 6,020,330, 7,179,792, EP251315, EP417725, EP489-423, EP549967, EP573848, EP624593, EP624594, EP624595, EP869121, EP1070703, WO04/005247, compounds disclosed as having IBAT activity in Drugs of the Future, 24, 425-430 (1999), Journal of Medicinal Chemistry, 48, 5837-5852, (2005) and Current Medicinal Chemistry, 13, 997-1016, (2006). In some embodiments, compounds that inhibit ASBT or any recuperative bile acid transporter are benzothiepines (including 1,2-benzothiazepines; 1,4-benzothiazepines; 1,5-benzothiazepines; and/or 1,2,5-benzothiadiazepines). In some embodiments, compounds that inhibit ASBT or any recuperative bile acid transporter include and are not limited to S-8921 (disclosed in EP597107, WO 93/08155), 264W94 (GSK) disclosed in WO 96/05188; SC-435 (1-[4-[4-[(4R,5R)-3,3-dibutyl-7-(dimethylamino)-2,3,4,5-tetrahydro-4-hydroxy-1,1-dioxido-1-benzothiepin-5-yl]phenoxy]butyl]-4-aza-1-azoniabicyclo[2.2.2]octane methanesulfonate salt), SC-635 (Searle); 2164U90 (3-butyl-3-ethyl-2,3,4,5-tetrahydro-5-phenyl-1,4-benzothiazepine 1,1-dioxide); BARI-1741 (Aventis SA), AZD 7508 (Astra Zeneca); barixibat (11-(D-gluconamido)-N-{2-[(1S,2R,3S)-3-hydroxy-3-phenyl-2-(2-pyridyl)-1-(2-pyridylamino)propyl]phenyl}undecanamide) or the like, or combinations thereof.

In some instances, inhibition of DPP-IV reduces the degradation of enteroendocrine peptide products (e.g. GLP-1) thereby prolonging the delay in gastric emptying and thereby reducing food intake. In some embodiments, the administration of bile acid sequestrant (e.g., labile bile acid sequestrant) described herein avoids weight gain that is associated with biguanide therapy and/or treatment with a DPP-IV inhibitor and/or a TGR5 agonist. In some embodiments, provided herein is administration of a GPR119 agonist, a GPR120 agonist, a GPR40 agonist, a GPR43 agonist, or a GPR154 agonist.

In some instances, the additional therapeutic agent modulates bile acid receptors in the gastrointestinal lumen. In some embodiments, the additional therapeutic agent agonizes or partially agonizes bile acid receptors (e.g., TGR5 receptors or Farnesoid-X receptors) in the gastrointestinal tract. In certain instances the additional therapeutic agent is a TGR5 agonist. In certain instances, administration of a TGR5 agonist in combination with any of the compounds described herein enhances the secretion of enteroendocrine peptides from L-cells. TGR5 modulators (e.g., agonists) include, and are not limited to, the compounds described in, WO 2008/091540, WO 2008067219 and US Appl. No. 2008/0221161, which are incorporated by reference herein for such compounds.

In some embodiments, the additional therapeutic agent is a biguanide. In some instances, biguanides reduce blood and/or plasma glucose levels. Examples of biguanides include and are not limited to metformin, buformin, phenformin, proguanil or the like.

In some embodiments, the additional therapeutic agent is an incretin mimetic. In some embodiments, an incretic mimic augments pancreas response to ingestion of food, In some instances, administration of an incretin mimetic in combination with any of the compounds described herein lowers blood and/or plasma glucose levels. Examples of incretin mimetics include and are not limited to exenatide (Byetta®).

One currently used therapy for the treatment of diabetes is a subcutaneous injection of exenatide (Byetta®). In some embodiments, an oral combination of a bile acid sequestrant (e.g., labile bile acid sequestrant) and a DPP-IV inhibitor is equally or more effective than an injection of exenatide in reducing plasma glucose levels. In some embodiments, an oral combination of a bile acid sequestrant (e.g., labile bile acid sequestrant) and a DPP-IV inhibitor reduces or eliminates discomfort associated with injections of glucose-lowering medications.

In some embodiments, the additional therapeutic agent is a thiazolidinedione. In some instances thiazolidinediones reverse insulin resistance and lower blood and/or plasma glucose levels. Examples of thiazolidinediones include and are not limited to Rosiglitazone (Avandia), Pioglitazone (Actos), Troglitazone (Rezulin), MCC-555, rivoglitazone, ciglitazone or the like.

Another therapy that is current standard of care for the treatment of diabetes is a combination of metformin and sitagliptin (Janumet®). In some embodiments, a combination of a bile acid sequestrant (e.g., labile bile acid sequestrant) and sitagliptin maintains reduced plasma glucose concentrations for a longer duration of time (e.g., at least 24 hours) compared to a combination of metformin and sitagliptin (about 6 hours). In some embodiments, a combination of a bile acid sequestrant (e.g., labile bile acid sequestrant) and sitagliptin reduces plasma glucose levels at a lower dose of a bile acid sequestrant (e.g., labile bile acid sequestrant) compared to the dose of metformin. In some instances a bile acid sequestrant (e.g., labile bile acid sequestrant) therapy eliminates side effects associated with metformin therapy.

DPP-IV inhibitors suitable for use with the methods described herein include and are not limited to (2S)-1-{2-[(3-hydroxy-1-adamantyl)amino]acetyl}pyrrolidine-2-carbonitrile (vildagliptin), (3R)-3-amino-1-[9-(trifluoromethyl)-1,4,7,8-tetrazabicyclo[4.3.0]nona-6,8-d ien-4-yl]-4-(2,4,5-trifluorophenyl)butan-1-one (sitagliptin), (1S,3S,5S)-2-[(2S)-2-amino-2-(3-hydroxy-1-adamantyl)acetyl]-2-azabicyclo[3.1.0]hexane-3-carbonitrile (saxagliptin), and 2-({6-[(3R)-3-aminopiperidin-1-yl]-3-methyl-2,4-dioxo-3,4-dihydropyrimidin-1(2H)-yl}methyl)benzonitrile (alogliptin).

In certain embodiments of any of the methods described herein, administration of a bile acid sequestrant (e.g., labile bile acid sequestrant) alone or in combination with a DPP-IV inhibitor reduces blood and/or plasma sugar levels by at least 20%, at least 30%, at least 40%, at least 50% at least 60%, at least 70% or at least 80% compared to blood and/or plasma sugar levels prior to administration of the a bile acid sequestrant (e.g., labile bile acid sequestrant) alone or in combination with a DPP-IV inhibitor. In some embodiments of any of the methods described herein, administration of a bile acid sequestrant (e.g., labile bile acid sequestrant) alone or in combination with a DPP-IV inhibitor reduces blood and/or plasma sugar levels by at least 20% compared to blood and/or plasma sugar levels prior to administration of the bile acid sequestrant (e.g., labile bile acid sequestrant) in combination with a DPP-IV inhibitor. In some embodiments of any of the methods described herein, administration of a bile acid sequestrant (e.g., labile bile acid sequestrant) alone or in combination with a DPP-IV inhibitor reduces blood and/or plasma sugar levels by at least 30% compared to blood and/or plasma sugar levels prior to administration of the bile acid sequestrant (e.g., labile bile acid sequestrant) in combination with a DPP-IV inhibitor. In some embodiments of any of the methods described herein, administration of a bile acid sequestrant (e.g., labile bile acid sequestrant) alone or in combination with a DPP-IV inhibitor reduces blood and/or plasma sugar levels by at least 40% compared to blood and/or plasma sugar levels prior to administration of the bile acid sequestrant (e.g., labile bile acid sequestrant) in combination with a DPP-IV inhibitor.

In some embodiments of any of the methods described herein, administration of a bile acid sequestrant (e.g., labile bile acid sequestrant) alone or in combination with a DPP-IV inhibitor reduces blood and/or plasma sugar levels for a longer period of time (e.g., at least 24 hours) compared to reduction in blood and/or plasma sugar levels upon administration of metformin in combination with a DPP-IV inhibitor. In some embodiments of any of the methods described herein, administration of a single dose of a bile acid sequestrant (e.g., labile bile acid sequestrant) alone or in combination with a DPP-IV inhibitor sustains reduced blood and/or plasma sugar levels for at least 6 hours, at least 12 hours, at least 14 hours, at least 16 hours, at least 18 hours, at least 20 hours, at least 24 hours, at least 30 hours, at least 36 hours or at least 48 hours compared to reduction in blood and/or plasma sugar levels upon administration of a single dose of metformin in combination with a DPP-IV inhibitor.

In some embodiments of any of the methods described herein, administration of a bile acid sequestrant (e.g., labile bile acid sequestrant) alone or in combination with a DPP-IV inhibitor results in higher levels of GLP-1 in blood and/or plasma of an individual compared to levels of GLP-1 in blood and/or plasma of a normal individual.

In some embodiments, a bile acid sequestrant (e.g., labile bile acid sequestrant) is administered in combination with a DPP-IV inhibitor and/or a biliary shunt. Examples of biliary shunts include and are not limited to the shunts described in WO 2007/0050628, the disclosure of biliary shunts described therein is incorporated herein by reference. In some of such embodiments, a biliary shunt moves bile acid to the distal ileum and/or the rectum and/or the colon thereby increasing the concentration of bile acids in the vicinity of L-cells present in the distal portion of the gastrointestinal tract. In some instances such an increase in the concentration of bile acids in the vicinity of L-cells stimulates the L-cells (e.g., increases the secretion of GLP-1 from L-cells) thereby inducing satiety and/or reduction in hunger and/or weight loss and/or reduction in plasma glucose levels or any combination thereof.

The combined use of a bile acid sequestrant (e.g., labile bile acid sequestrant) alone or in combination with a second active ingredient are used such that the bile acid sequestrant or the combination is present in a therapeutically effective amount. That therapeutically effective amount may arise from the use of a combination of a bile acid sequestrant (e.g., labile bile acid sequestrant) and the other active ingredient (e.g., a DPP-IV inhibitor) wherein each is used in a therapeutically effective amount, or by virtue of additive or synergistic effects arising from the combined use, each can also be used in a subclinical therapeutically effective amount, i.e., an amount that, if used alone, provides for reduced effectiveness for the therapeutic purposes noted herein, provided that the combined use is therapeutically effective. In some embodiments, the use of a combination of a bile acid sequestrant (e.g., labile bile acid sequestrant) and any other active ingredient as described herein encompasses combinations where the bile acid sequestrant (e.g., labile bile acid sequestrant) or the other active ingredient is present in a therapeutically effective amount, and the other is present in a subclinical therapeutically effective amount, provided that the combined use is therapeutically effective owing to their additive or synergistic effects. As used herein, the term “additive effect” describes the combined effect of two (or more) pharmaceutically active agents that is equal to the sum of the effect of each agent given alone. A synergistic effect is one in which the combined effect of two (or more) pharmaceutically active agents is greater than the sum of the effect of each agent given alone. Any suitable combination of a bile acid sequestrant (e.g., labile bile acid sequestrant) with one or more of the aforementioned other active ingredients and optionally with one or more other pharmacologically active substances is contemplated as being within the scope of the methods described herein.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g, from more than zero weeks to less than four weeks. In some embodiments, the additional therapeutic agent is utilized to achieve remission (partial or complete) of a cancer, whereupon the therapeutic agent described herein is subsequently administered. In addition, the combination methods, compositions and formulations are not to be limited to the use of only two agents; the use of multiple therapeutic combinations are also envisioned (including two or more compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy described herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In certain embodiments, a bile acid sequestrant (e.g., labile bile acid sequestrant) described herein is combined with or utilized in combination with one or more of the following therapeutic agents in any combination: ASBT inhibitor, insulin, insulin-mimetics, incretin mimetics, GLP-1, GLP-2, oxyntomodulin, PYY, DPP-IV inhibitors, TGR5 modulators, a GPR119 agonist, a GPR120 agonist, a GPR40 agonist, a GPR43 agonist, or a GPR154 agonist.

In certain embodiments the bile acid sequestrants (e.g., labile bile acid sequestrants) or compositions described herein are used in combination (in a combination therapy and/or formulation) with at least one appetite suppressant (e.g., a 5HT transport inhibitor, a NE transport inhibitor, a CB-1 antagonist/inverse agonist, a ghrelin antagonist, a H3 antagonist/inverse agonist, a MCH1R antagonist, a MCH2R agonist/antagonist, a NPY1 antagonist, a NPY2 agonist, a mGluR5 antagonist, leptin, a leptin agonist/modulator, a leptin derivative, amylin, symlin, a symlin analogue, an amylin agonist/modulator, an opiod antagonist, an orexin antagonist, a BRS3 agonist, a CCK-A agonist, CNTF, a CNTF agonist/modulator, a CNTF derivative, a 5HT2c agonist, a Mc5r agonist, a monoamine reuptake inhibitor, a serotonin reuptake inhibitor, a GLP-1 agonist, axokine, fenfluramine, nalmafene, phentermine, rimonabant, sibutramine, topiramate, phytopharm compound 57, and combinations thereof). In certain embodiments the compositions or formulations described herein are used in combination with at least one metabolic rate enhancing agents (e.g., an ACC2 inhibitor, a β3 agonist, DGAT1 inhibitor, a DGAT2 inhibitor, a FAS inhibitor, a PDE inhibitor, a thyroid hormone β agonist, an UCP-1, 2, or 3 activator, an acyl-estrogen, a glucocorticoid antagonist, an 11 HSD-1 inhibitor, a Mc3r agonist, a SCD-1, oleoyl-estrone, 3-[(3,5,7-trimethyl-1-adamantyl)methyl]-6,7,8,9-tetr-ahydro-5H-[1,2,4]triazolo[4,3-a]azepine; 3-(1-adamantyl)-4-ethyl-5-(e-thylthio)-4H-1,2,4-triazole; 3-adamantanyl-4,5,6,7,8,9,10,11,12,3a-de-cahydro-1,2,4-triazolo[4,3-a][11]annulene, 3-(1-adamantyl)-5-(3,4,5-trimethoxyphenyl)-4-methyl-4H-1,2,4-triazole and combinations thereof). In some embodiments, an bile acid sequestrant is combined or administered with a phosphodiesterase inhibitor. In certain embodiments, an bile acid sequestrant is combined or administered with caffeine. In certain embodiments the compositions or formulations described herein are used in combination with at least one nutrient absorption inhibitors (e.g., a lipase inhibitor; a fatty acid transporter inhibitor; a dicarboxylate transporter inhibitor; a glucose transporter inhibitor; a phosphate transporter inhibitor; orlistat and combinations thereof). In certain embodiments the compositions or formulations described herein are used in combination with at least one appetite suppressant and at least one metabolic rate enhancing agents. In certain embodiments the compositions or formulations described herein are used in combination with at least one appetite suppressant and at least one nutrient absorption inhibitors. In certain embodiments the compositions or formulations described herein are used in combination with at least one nutrient absorption inhibitors and at least one metabolic rate enhancing agents. In certain embodiments the compositions or formulations described herein are used in combination with at least one appetite suppressant, at least one metabolic rate enhancing agents and at least one nutrient absorption inhibitors.

In some embodiments, a combination therapy described herein provides for administration of a bile acid sequestrant (e.g., labile bile acid sequestrant) and an appetite suppressant (e.g., leptin, a leptin agonist/modulator, a leptin derivative, amylin, symlin, a symlin analogue, and/or an amylin agonist/modulator). In specific embodiments, a combination therapy described herein comprises the administration of a bile acid sequestrant (e.g., labile bile acid sequestrant) and leptin, a leptin agonist/modulator, and/or a leptin derivative. In some specific embodiments, a combination therapy described herein comprises the administration of a bile acid sequestrant (e.g., labile bile acid sequestrant) and amylin, symlin, a symlin analogue, and/or an amylin agonist/modulator. In certain instances, such therapies provide for increases in amounts of multiple enteroendocrine peptide products that provide for an enhanced efficacy and/or enhanced duration of beneficial therapy. In some instances, therapy that involves administration and/or stimulation of a single enteroendocrine peptide product may result in a decrease in or elimination of efficacy over short periods of time. In certain instances, a combination therapy described herein (e.g., comprising administration and/or up-regulation of two or more enteroendocrine peptide products) provides greater efficacy and/or greater duration of efficacy (e.g., synergistic efficacy) than single therapy (e.g., comprising administration and/or up-regulation of a single enteroendocrine peptide product).

### Pharmaceutical Compositions

Provided herein, in certain embodiments, is a pharmaceutical composition comprising a therapeutically effective amount of a bile acid sequestrant (e.g., a labile bile acid sequestrant, such as one described herein).

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a compound described herein, with other pharmaceutically acceptable agents, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the compounds described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes.

A “carrier” includes, in some embodiments, a pharmaceutically acceptable excipient and is selected on the basis of compatibility with therapeutic agents, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein are formulated as a dosage form. As such, in some embodiments, provided herein is a dosage form comprising a compound described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation comprising the therapeutic agent or agents described herein. In one embodiment, a compound described herein is in the form of a particle and some or all of the particles of the compound are coated. In certain embodiments, some or all of the particles of a compound described herein are microencapsulated. In some embodiments, the particles of the compound described herein are not microencapsulated and are uncoated.

In some embodiments, a bile acid sequestrant (e.g., labile bile acid sequestrant) is used in the preparation of medicaments for the prophylactic and/or therapeutic treatment of obesity and/or diabetes. A method for treating any of the diseases or conditions described herein in an individual in need of such treatment, involves administration of pharmaceutical compositions containing at least one bile acid sequestrant (e.g., labile bile acid sequestrant) in therapeutically effective amounts to said individual.

In the case wherein the patient's condition does not improve, upon the doctor's discretion the administration of a bile acid sequestrant (e.g., labile bile acid sequestrant) is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of a bile acid sequestrant (e.g., labile bile acid sequestrant) is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms (e.g., weight gain).

What is claimed is:

1. A method of treating obesity or diabetes in an individual comprising administering to an individual in need thereof an effective amount of a labile bile acid sequestrant, wherein the labile bile acid sequestrant has a low affinity in the colon or rectum of a human for at least one bile acid or bile acid mimic that stimulates L-cells.

2. The method of claim 1, wherein the method is a method of treating obesity.

3. The method of claim 1, wherein the method is a method of treating diabetes.

4. The method of claim 1, wherein the method is a method of treating obesity and diabetes.

5. The method of claim 1, wherein the labile bile acid sequestrant is a non-systemic labile bile acid sequestrant.

6. The method of claim 3, wherein the non-systemic labile bile acid sequestrant is absorbed less than 10% systemically.

7. The method of claim 1, wherein the labile bile acid sequestrant is conjugated to an exogenous bile acid, a bile salt, a bile acid mimic, a free fatty acid, or a free fatty acid mimic.

8. The method of claim 1, wherein the labile bile acid sequestrant is an enzyme-dependent bile acid sequestrant.

9. The method of claim 8, wherein the enzyme is a bacterial enzyme found in high concentrations in human colon or rectum relative to the concentration found in the small intestine.

10. The method of claim 1, wherein the labile bile acid sequestrant is a time-dependent bile acid sequestrant.

11. The method of claim 1, wherein the labile bile acid sequestrant is a pH-dependent bile acid sequestrant.

12. The method of claim 11, wherein the pH-dependent bile acid sequestrant has a high affinity for bile acid at a pH of 7 or below and a low affinity for bile acid at a pH above 7.

13. The method of claim 1, wherein the labile bile acid sequestrant is lignin or a modified lignin.

14. The method of claim 1, wherein the labile bile acid sequestrant is a polymer or copolymer comprising one or more N-alkenyl-N-alkylamine residues; one or more N,N,N-trialkyl-N—(N′-alkenylamino)alkyl-azanium residues; one or more N,N,N-trialkyl-N-alkenyl-azanium residues; one or more alkenyl-amine residues; or a combination thereof.

15. The method of claim 1, wherein levels of GLP-1 in the blood or plasma of the individual are increased by about 2 times to about 6 times the level of GLP-1 in the blood or plasma of the individual prior to administration of the bile acid sequestrant.

16. The method of claim 1, wherein levels of post-prandial glucose in the blood or plasma of the individual are reduced by at least 30% compared to the level of glucose in the blood or plasma of the individual prior to administration of the bile acid sequestrant.

17. The method of claim 1, wherein the labile bile acid sequestrant transports bile acids from the duodenum, the jejunum, or the ileum of the individual to the colon or the rectum of the individual.

18. The method of claim 1, wherein the bile acid sequestrant is a carrier or a delivery agent of a bile acid, a bile salt, a bile acid mimic, or a bile salt mimic, free fatty acids, or a free fatty acids mimics.

19. The method of claim 1, wherein the effective amount of bile acid sequestrant is an amount sufficient to provide a bile acid concentration in the colon of greater than 3 mM.

20. The method of claim 1, further comprising administration of a second agent selected from an ASBT inhibitor, metformin, an incretin mimetic, a DPP-IV inhibitor, a TGR5 agonist, a GPR119 agonist, a GPR120 agonist, a GPR40 agonist, a GPR43 agonist, and a GPR154 agonist.
